SEQUENCE LISTING (13 sequences)

SEQ ID NO: 1 - DNA, 1098 bp, Homo sapiens
  source 1..1098: mol_type = genomic DNA
atgctggccgtcggctgcgcgctgctggctgccctgctggccgcgccgggagcggcgctggccccaaggcgctgccctgcgcaggaggtggcgagaggcgtgctgaccagtctgccaggagacagcgtgactctgacctgcccgggggtagagccggaagacaatgccactgttcactgggtgctcaggaagccggctgcaggctcccaccccagcagatgggctggcatgggaaggaggctgctgctgaggtcggtgcagctccacgactctggaaactattcatgctaccgggccggccgcccagctgggactgtgcacttgctggtggatgttccccccgaggagccccagctctcctgcttccggaagagccccctcagcaatgttgtttgtgagtggggtcctcggagcaccccatccctgacgacaaaggctgtgctcttggtgaggaagtttcagaacagtccggccgaagacttccaggagccgtgccagtattcccaggagtcccagaagttctcctgccagttagcagtcccggagggagacagctctttctacatagtgtccatgtgcgtcgccagtagtgtcgggagcaagttcagcaaaactcaaacctttcagggttgtggaatcttgcagcctgatccgcctgccaacatcacagtcactgccgtggccagaaacccccgctggctcagtgtcacctggcaagacccccactcctggaactcatctttctacagactacggtttgagctcagatatcgggctgaacggtcaaagacattcacaacatggatggtcaaggacctccagcatcactgtgtcatccacgacgcctggagcggcctgaggcacgtggtgcagcttcgtgcccaggaggagttcgggcaaggcgagtggagcgagtggagcccggaggccatgggcacgccttggacagaatccaggagtcctccagctgagaacgaggtgtccacccccatgcaggcacttactactaataaagacgatgataatattctcttcagagattctgcaaatgcgacaagcctcccaggttcaagaagacgtggaagctgcgggctctga

SEQ ID NO: 2 - protein, 244 aa, Homo sapiens
VPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLP

SEQ ID NO: 3 - DNA, 636 bp, Homo sapiens
  source 1..636: mol_type = genomic DNA
atgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatg

SEQ ID NO: 4 - protein, 543 aa, synthetic construct
  REGION 1..543: note = IL6RIL6 chimera amino acid sequence
MLAVGCALLAALLAAPGAALAPRRCPAQEVARGVLTSLPGDSVTLTCPGVEPEDNATVHWVLRKPAAGSHPSRWAGMGRRLLLRSVQLHDSGNYSCYRAGRPAGTVHLLVDVPPEEPQLSCFRKSPLSNVVCEWGPRSTPSLTTKAVLLVRKFQNSPAEDFQEPCQYSQESQKFSCQLAVPEGDSSFYIVSMCVASSVGSKFSKTQTFQGCGILQPDPPANITVTAVARNPRWLSVTWQDPHSWNSSFYRLRFELRYRAERSKTFTTWMVKDLQHHCVIHDAWSGLRHVVQLRAQEEFGQGEWSEWSPEAMGTPWTESRSPPAENEVSTPMQALTTNKDDDNILFRDSANATSLPVEFMPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 5 - DNA, 2988 bp, Homo sapiens
  source 1..2988: mol_type = genomic DNA
gcaggagggcccagcgacgccgccgcgccagctcccagggcccggccccccccggcgctcacgctctcggggcggactcccggccctccgcgccctctcgcgcggcgatggccccactcggatacttcttactcctctgcagcctgaagcaggctctgggcagctacccgatctggtggtcgctggctgttgggccacagtattcctccctgggctcgcagcccatcctgtgtgccagcatcccgggcctggtccccaagcagctccgcttctgcaggaactacgtggagatcatgcccagcgtggccgagggcatcaagattggcatccaggagtgccagcaccagttccgcggccgccggtggaactgcaccaccgtccacgacagcctggccatcttcgggcccgtgctggacaaagctaccagggagtcggcctttgtccacgccattgcctcagccggtgtggcctttgcagtgacacgctcatgtgcagaaggcacggccgccatctgtggctgcagcagccgccaccagggctcaccaggcaagggctggaagtggggtggctgtagcgaggacatcgagtttggtgggatggtgtctcgggagttcgccgacgcccgggagaaccggccagatgcccgctcagccatgaaccgccacaacaacgaggctgggcgccaggccatcgccagccacatgcacctcaagtgcaagtgccacgggctgtcgggcagctgcgaggtgaagacatgctggtggtcgcaacccgacttccgcgccatcggtgacttcctcaaggacaagtacgacagcgcctcggagatggtggtggagaagcaccgggagtcccgcggctgggtggagaccctgcggccgcgctacacctacttcaaggtgcccacggagcgcgacctggtctactacgaggcctcgcccaacttctgcgagcccaaccctgagacgggctccttcggcacgcgcgaccgcacctgcaacgtcagctcgcacggcatcgacggctgcgacctgctgtgctgcggccgcggccacaacgcgcgagcggagcggcgccgggagaagtgccgctgcgtgttccactggtgctgctacgtcagctgccaggagtgcacgcgcgtctacgacgtgcacacctgcaagtaggcaccggccgcggctccccctggacggggcgggccctgcctgagggtgggcttttccctgggtggagcaggactcccacctaaacggggcagtactcctccctgggggcgggactcctccctgggggtggggctcctacctgggggcagaactcctacctgaaggcagggctcctccctggagctagtgtctcctctctggtggctgggctgctcctgaatgaggcggagctccaggatggggaggggctctgcgttggcttctccctggggacggggctcccctggacagaggcggggctacagattgggcggggcttctcttgggtgggacagggcttctcctgcgggggcgaggcccctcccagtaagggcgtggctctgggtgggcggggcactaggtaggcttctacctgcaggcggggctcctcctgaaggaggcggggctctaggatggggcacggctctggggtaggctgctccctgagggcggagcgcctccttaggagtggggttttatggtggatgaggcttcttcctggatggggcagagcttctcctgaccagggcaaggccccttccacgggggctgtggctctgggtgggcgtggcctgcataggctccttcctgtgggtggggcttctctgggaccaggctccaatggggcggggcttctctccgcgggtgggactcttccctgggaaccgccctcctgattaaggcgtggcttctgcaggaatcccggctccagagcaggaaattcagcccaccagccacctcatccccaaccccctgtaaggttccatccacccctgcgtcgagctgggaaggttccatgaagcgagtcgggtccccaacccgtgcccctgggatccgagggcccctctccaagcgcctggctttggaatgctccaggcgcgccgacgcctgtgccaccccttcctcagcctggggtttgaccacccacctgaccaggggccctacctggggaaagcctgaagggcctcccagcccccaaccccaagaccaagcttagtcctgggagaggacagggacttcgcagaggcaagcgaccgaggccctcccaaagaggcccgccctgcccgggctcccacaccgtcaggtactcctgccagggaactggcctgctgcgccccaggccccgcccgtctctgctctgctcagctgcgcccccttctttgcagctgcccagcccctcctccctgccctcgggtctccccacctgcactccatccagctacaggagagatagaagcctctcgtcccgtccctccctttcctccgcctgtccacagccccttaagggaaaggtaggaagagaggtccagccccccaggctgcccagagctgctggtctcatttgggggcgttcgggaggtttggggggcatcaaccccccgactgtgctgctcgcgaaggtcccacagccctgagatgggccggcccccttcctggcccctcatggcgggactggagaaatggtccgctttcctggagccaatggcccggcccctcctgactcatccgcctggcccgggaatgaatggggaggccgctgaacccacccggcccatatccctggttgcctcatggccagcgcccctcagcctctgccactgtgaaccggctcccaccctcaaggtgcggggagaagaagcggccaggcggggcgccccaagagcccaaaagagggcacaccgccatcctctgcctcaaattctgcgtttttggttttaatgttatatctgatgctgctatatccactgtccaacggagttagacgaaaaaaaaaaaaaaaaaa

SEQ ID NO: 6 - protein, 352 aa, Homo sapiens
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 7 - protein, 288 aa, Homo sapiens
MVGVGGGDVEDVTPRPGGCQISGRGARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 8 - protein, 202 aa, Homo sapiens
MKVLAAGVVPLLLVLHWKHGAGSPLPITPVNATCAIRHPCHNNLMNQIRSQLAQLNGSANALFILYYTAQGEPFPNNLDKLCGPNVTDFPPFHANGTEKAKLVELYRIVVYLGTSLGNITRDQKILNPSALSLHSKLNATADILRGLLSNVLCRLCSKYHVGHVDVTYGPDTSGKDVFQKKKLGCQLLGKYKQIIAVLAQAF

SEQ ID NO: 9 - DNA, 3987 bp, Homo sapiens
  source 1..3987: mol_type = genomic DNA
cttcctggactggggatcccggctaaatatagctgtttctgtcttacaacacaggctccagtatataaatcaggcaaattccccatttgagcatgaacctctgaaaactgccggcatctgaggtttcctccaaggccctctgaagtgcagcccataatgaaggtcttggcggcaggagttgtgcccctgctgttggttctgcactggaaacatggggcggggagccccctccccatcacccctgtcaacgccacctgtgccatacgccacccatgtcacaacaacctcatgaaccagatcaggagccaactggcacagctcaatggcagtgccaatgccctctttattctctattacacagcccagggggagccgttccccaacaacctggacaagctatgtggccccaacgtgacggacttcccgcccttccacgccaacggcacggagaaggccaagctggtggagctgtaccgcatagtcgtgtaccttggcacctccctgggcaacatcacccgggaccagaagatcctcaaccccagtgccctcagcctccacagcaagctcaacgccaccgccgacatcctgcgaggcctccttagcaacgtgctgtgccgcctgtgcagcaagtaccacgtgggccatgtggacgtgacctacggccctgacacctcgggtaaggatgtcttccagaagaagaagctgggctgtcaactcctggggaagtataagcagatcatcgccgtgttggcccaggccttctagcaggaggtcttgaagtgtgctgtgaaccgagggatctcaggagttgggtccagatgtgggggcctgtccaagggtggctggggcccagggcatcgctaaacccaaatgggggctgctggcagaccccgagggtgcctggccagtccactccactctgggctgggctgtgatgaagctgagcagagtggaaacttccatagggagggagctagaagaaggtgccccttcctctgggagattgtggactggggagcgtgggctggacttctgcctctacttgtccctttggccccttgctcactttgtgcagtgaacaaactacacaagtcatctacaagagccctgaccacagggtgagacagcagggcccaggggagtggaccagcccccagcaaattatcaccatctgtgcctttgctgccccttaggttgggacttaggtgggccagaggggctaggatcccaaaggactccttgtcccctagaagtttgatgagtggaagatagagaggggcctctgggatggaaggctgtcttcttttgaggatgatcagagaacttgggcataggaacaatctggcagaagtttccagaaggaggtcacttggcattcaggctcttggggaggcagagaagccaccttcaggcctgggaaggaagacactgggaggaggagaggcctggaaagctttggtaggttcttcgttctcttccccgtgatcttccctgcagcctgggatggccagggtctgatggctggacctgcagcaggggtttgtggaggtgggtagggcaggggcaggttgctaagtcaggtgcagaggttctgagggacccaggctcttcctctgggtaaaggtctgtaagaaggggctggggtagctcagagtagcagctcacatctgaggccctgggaggccttgtgaggtcacacagaggtacttgagggggactggaggccgtctctggtccccagggcaagggaacagcagaacttagggtcagggtctcagggaaccctgagctccaagcgtgctgtgcgtctgacctggcatgatttctatttattatgatatcctatttatattaacttattggtgctttcagtggccaagttaattcccctttccctggtccctactcaacaaaatatgatgatggctcccgacacaagcgccagggccagggcttagcagggcctggtctggaagtcgacaatgttacaagtggaataagccttacgggtgaagctcagagaagggtcggatctgagagaatggggaggcctgagtgggagtggggggccttgctccacccccccccatcccctactgtgacttgctttagggtgtcagggtccaggctgcaggggctgggccaatttgtggagaggccgggtgcctttctgtcttgattccagggggctggttcacactgttcttgggcgccccagcattgtgttgtgaggcgcactgttcctggcagatattgtgccccctggagcagtgggcaagacagtccttgtggcccaccctgtccttgtttctgtgtccccatgctgcctctgaaatagcgccctggaacaaccctgcccctgcacccagcatgctccgacacagcagggaagctcctcctgtggcccggacacccatagacggtgcggggggcctggctgggccagaccccaggaaggtggggtagactggggggatcagctgcccattgctcccaagaggaggagagggaggctgcagatgcctgggactcagaccaggaagctgtgggccctcctgctccacccccatcccactcccacccatgtctgggctcccaggcagggaacccgatctcttcctttgtgctggggccaggcgagtggagaaacgccctccagtctgagagcaggggagggaaggaggcagcagagttggggcagctgctcagagcagtgttctggcttcttctcaaaccctgagcgggctgccggcctccaagttcctccgacaagatgatggtactaattatggtacttttcactcactttgcacctttccctgtcgctctctaagcactttacctggatggcgcgtgggcagtgtgcaggcaggtcctgaggcctggggttggggtggagggtgcggcccggagttgtccatctgtccatcccaacagcaagacgaggatgtggctgttgagatgtgggccacactcacccttgtccaggatgcagggactgccttctccttcctgcttcatccggcttagcttggggctggctgcattcccccaggatgggcttcgagaaagacaaacttgtctggaaaccagagttgctgattccacccggggggcccggctgactcgcccatcacctcatctccctgtggacttgggagctctgtgccaggcccaccttgcggccctggctctgagtcgctctcccacccagcctggacttggccccatgggacccatcctcagtgctccctccagatcccgtccggcagcttggcgtccaccctgcacagcatcactgaatcacagagcctttgcgtgaaacagctctgccaggccgggagctgggtttctcttccctttttatctgctggtgtggaccacacctgggcctggccggaggaagagagagtttaccaagagagatgtctccgggcccttatttattatttaaacatttttttaaaaagcactgctagtttacttgtctctcctccccatcgtccccatcgtcctccttgtccctgacttggggcacttccaccctgacccagccagtccagctctgccttgccggctctccagagtagacatagtgtgtggggttggagctctggcacccggggaggtagcatttccctgcagatggtacagatgttcctgccttagagtcatctctagttccccacctcaatcccggcatccagccttcagtcccgcccacgtgctagctccgtgggcccaccgtgcggccttagaggtttccctccttcctttccactgaaaagcacatggccttgggtgacaaattcctctttgatgaatgtaccctgtggggatgtttcatactgacagattatttttatttattcaatgtcatatttaaaatatttattttttataccaaatgaatacttttttttttaagaaaaaaaagagaaatgaataaagaatctactcttggctggcaaaaaaaaaaaaaaaaaa

SEQ ID NO: 10 - protein, 202 aa, Bos taurus
MKVLAAGVVPLLLVLHWKHGAGSPLPITPVNATCATRHPCPSNLMNQIRNQLGQLNSSANSLFILYYTAQGEPFPNNLDKLCSPNVTDFPPFHANATEKARLVELYRIIAYLGASLGNITRDQKVLNPYAHGLHSKLSTTADVLRGLLSNVLCRLCSKYHVSHVDVTYGPDTSGKDVFQKKKLGCQLLGKYKQVIAVLAQAF

SEQ ID NO: 11 - DNA, 1831 bp, Bos taurus
  source 1..1831: mol_type = genomic DNA
ggatccctgctaaatatagctgtttctctctctgtcttacaacacaggctccagtatataaatcaggcaaattccccatttgagcatgaacctctgaaaactgccggcatctaaggtctccttcaaggccctctggagtgcagcccataatgaaggtcttggcggcaggagttgtgcccttgctgctggttctccactggaaacacggggccgggagcccccttcccatcaccccggtcaacgccacctgtgccacccgccatccctgtcccagcaacctcatgaaccagatcagaaaccagctgggacaactcaacagcagtgccaacagcctctttatcctctattacacggcccagggggagcccttccccaacaacctggacaagctgtgcagccccaacgtgactgacttcccgcccttccacgccaacgcgacggagaaggcccggctggtggagctgtaccgcatcatcgcgtacctgggcgcctccctgggcaacatcacgagagaccagaaggtcctcaacccctacgcccacggcctgcacagcaagctgagcaccacggccgacgtcctgcggggtctgctcagcaacgtgctctgccgcttgtgcagcaagtaccacgtgagccacgtggacgtgacctacggccccgacacctcgggcaaggacgtcttccagaagaagaagctgggctgtcagctcctggggaagtacaagcaggtcatcgccgtgctggcccaggccttctagacgggaggtcttagatagtaggggactctccaactgcagccgtggcccagagcactgccagacccgagtaggggccgctggcagacccctgaggggttcctggccggtccactcccctccagggtgggccgccacgaagccgagcagagccagaactcccagaggcagaacctatacgtggtgccaactagaaaggaaggcgccccttcttctgggagactacagccgggcacgcagtgtcgggctggagtttggcccctgactcatcccctcagccagggtctttgtgagcaaaccccgaaagttgtctctggcgaccctgaccacggggtgagacagcaggggtcgggggcactaacccgcgaccccccagcagaatgaccaccatcagtgccttggctgaccttgaaaggtctggttggagctcaggcagcctggaggggctgggatccggaaggacccttggctcctacaggtatggcgagttggaagtctagaacgggagctgtggtttgagatgctgccttgcttgggcaagactggggagttcaggcctatagctctcaggtggaaggttccagaaggaggccacatggcctccaggctctcggggaggcagagagaggccacatgtaggccggggaaggaagagaggcccttgaaagtcttgacgggctccccttccttgagcccaaagtctgtcctgccatccttgctggccgcggggtctagggactggacctggaggcggggtgtgtgggggcgggcaggttgcaaagtcaggtgaggaggttctaaggggatccagagtcttgtgggcgagggtgtgcggatggagtaggtcagagtggcagctcccgtctgaggcccaccgaggcccggtgaggtcacacagaggtacgccgggggcaccagaggccatctgtcgctggttcctggagcagggggacggtgggagttgaggtcagggtctcagaagcctgagagccaagagtgctgtgtgcctgactcagcatgattgtctatttattttgatgccctatttatattaacttattggtgcttcaaatggc

SEQ ID NO: 12 - protein, 155 aa, Bos taurus
MAAGSITTLPSLPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYSSWYVALKRTGQYKLGPKTGPGQKAILFLPMSAKS

SEQ ID NO: 13 - DNA, 6594 bp, Bos taurus
  source 1..6594: mol_type = genomic DNA
ccggggccgcgccgcggagcgcgtcggaggccggggccggggcgcggcggctccccgcgcggctccaggggctcggggaccccgccagggccttggtggggccatggccgccgggagcatcaccacgctgccatccctgccggaggacggcggcagcggcgctttcccgccgggccacttcaaggaccccaagcggctgtactgcaagaacgggggcttcttcctgcgcatccaccccgacggccgagtggacggggtccgcgagaagagcgacccacacatcaaactacaacttcaagcagaagagagaggggttgtgtctatcaaaggagtgtgtgcaaaccgttaccttgctatgaaagaagatggaagattactagcttctaaatgtgttacagacgagtgtttcttttttgaacgattggagtctaataactacaatacttaccggtcaaggaaatactccagttggtatgtggcactgaaacgaactgggcagtataaacttggacccaaaacaggacctgggcagaaagctatactttttcttccaatgtctgctaagagctgatcttaatggcagcatctgatctcattttacatgaagaggtatatttcagaaatgtgttaatgaaaaaagaaaaatgtgtacagtgagctgctcagtttgggtaactgttcagataaccgtttatctaagagtaaaatatttaaccattgccttagtttttttttaaagaaaaaacacaataacagcaaaaattcctggaaaatgtatacatttccactttttatacagcatttccttttatccagtgaaacttacttaaagctacaatctttcatacagttgcttcatttgaagaggcttttaaaatgtgtacaaacaagttttcttcatggaaattatagacattagaaaattaaagtcatatttagttattaacccaaatgtccactacttcctataatatggcacacattaatctacatgtacaacttacttaaacatgtacaacttacttaaacattttaaaaacatgtaaatatgaatttaatccattcctgtcatagttttgtaattgtctggcagtttcttgtgatagagtttatagaacaagcctgtgtaaactgctggcagttcttccatggtcagatcaattttgtcaaacccttctttgtacccatacagcagcagccttgcaactctgcttgttatgggagtcgtatttttagtcttgactagatcgctgagattcatccactcacactttaagcattcacgctggcaaaaatttatggtgaatgaatatggctttaagcggcagataatatacatatctgacttcccaaaagctcctggatgggtgtgctgttgccgaatactcaggagggatctgaattcggattttataccagtctcttcaaaaacttctcgaactgctgtatctcctacataaaagaaaatgtacaaatcaataacgattatacttttagaaatttaatcaaagattttcagataaggaagcattattatgtaaagattcaaaaggtaaaaatttaccctaagaaaagaaagctttccctgtaaactctgtcctctggacatcctgaaaaaacaaagtattttcttaccactgtatagctaagaagcttttgaaataatatttctttggcttctacttgcaagcttacccatctatatatatgtattttgggagtcacatatttttaaattcttcctgctttatttcccaaaagttaatattcctgtatattttttcattattatcttgttcctgattatccattaaaactgcctaaactgataaacatttgaagtaagaaaaagtgatccattcttctttacaaaagtctgtagagctgcagaatatatagaactaggaaatgattcaaatcatccctggtctctcctgggactgtcaggcctctgaagtcataggtcggatttcgttataaccattttgttatgctcttctagttattctgtcagtggaatcccaccatggtaatttctggcattttctttgtttcttgctgtttcaaagaacttggattcattcttctaacaccaaaatgctacagtcatcagaagtttaaaaaaaaacttgcaatttacagaattttataatattaccaggcttttcacattttataaagttgatttttaaataatatgcaaatttctaggacaggatttttattgccattaacttatttttgtggctgctctttctaaatatccagatgaacctcctacctgggatttctgtaattttctgatgctgtcattgtctcccaaagtgtttatgaaaagccctaaaaaagctgccttccttgtctattttctgggaagtttcacaattgccacaagtatagatttttgtttaaatatcttttaatgccttcattttcttgtttgtcaggttgtaaactgtatttggcttctcagtagtcctgctagtgaggaataggcaaggaagagcaagtaaacaagaaatgttgcagtgttttttctaataacagctctggaaataagcacaggaagagtagtgtgtaaaatatgacatctgtctaccatatttgaattctgtgtgaacgaactttttaattgagatttgctaaagatcaaatcaacatggttagaaattatatttttaaactgaaaatatagaaaaatatatgttaagaaaaggaaaacttggcttaagaaaaataatttttgttgtattaaaaaacttgtattaagtttgttacagattgtggcactagtcttaaattttacatgtcatttgctgatctgacttaaaaattgttcaaatgtttaaaaagttctttaaacattttaaaatgaccatggggatcttgtttagctcttaataacactagtcaagagtttaacatttagttcctgtgtctagcctgcttgtatgttatagaagcacaggatggggctggtgagtgaatctgccaggcttagccatcaccacagcagctgattcaaaatcagcactgcctggatagtttgatccatttaacttgaatcatgatgtcattaactagattaaaaattaaatgggcaaataagtgcttttagatctagaggaaccaaccccttctatattaaaattgaaatctcttctccaaggattttatgatgaattaaaaattttaatttaggtaaagtgcgttatttgctggtattattttaaatgtactgtaagtaaactgaataacggttttatagatttgaagaatataggaaaaccaagagggttttgtttttatttttgctggttgaaagatgtttaaaaacatcatagtgttttatttagttaaaggacagtactgaaatggagtttatatttgttacttctattttgtaatatttaataacaggattaggttgaaataaaataataggaaaaactgtgcagaatgtggattttcctggtgtctccccctcactctggtacactgatgagctctgagcagaccccactgctttacagacctttggctatacagggagttctcttcctgttagtgctaatgagattttcccccccccagaaaggcagcttctgtttttaaccttatctatagataggcttatcggagaaggcaatggcaccccactccagaactcttgcctggaaaatcccatggatggaggagcctggtgggctgcagtccatggggtcgctaagagttggacacgactgagcgacttcactttcacttttcactttcatgcattggagaaggaaatggcaacccactccggtgttcttgcctggagaatccgggggacgagggagcctggtgagctgctgtctatggggtcgcagagtcggacatgactgaagtgacttagcagcagcatagatacctttttgtactctgcttcatttacctaatacttatcaaagaatgaaggattccaaacaaatgagcttcttattttaactagtatttactgcttaacagccagtatgaacatttgcacatttatgatggcggcagtcctattacatactttcctaaaaacagagtttaaagaaaataaataattcctggttgatttggcttcatcattaagagtaatctattactatactgttacaaaacagaaatgtactctacatagacatggtctttcagatctctatgtctcttatcatttctagctgctttcagagttttatcacttctgaggcaatgcttcagtttttcctactcctaggcaatatggtaaatgccagttgctgcttttttcttaattccatgtggctggaggcattaaaaacaatctctgactaggtgggttgttgttatacccacaagtatttttaaaaagtagtgaatttctagttatatggacttgaaatgttctggagtacactcaaacctaaagtgtacttatttacatggtgtggaaatgtgtttatttacatttaaatatatctgaaattcagaatatcaatgaaaactcaaatgaaaaaagttattcatttgaaagaaaaaaaaaaaaaaagttattcatttgagaaggcaaggttcagaagaggaagttatacaaacttcctatagactgctatttgcccagtatggattagataaggatgtaaaacagacacttaactagttcacatgatctcatatcacatgatagtgtgagataaccgggaattctagagtaaatggctttttctttcagcactggcactactacaaaatccttttatttcaacagaagacctagggaagactaagctaaaggtcagtgagcacctaaaaaccaaaatctgctatgatatatttgtagtgaaatttatttataggatgttaggagttggctgtatactacaaataggacattttcatctgtggaacattaaaaaaaaatcatttcaagtatatatatatacatttaaaaataatttagggcactgccttcatataaatgatggctaaagagaatagggtacatatacacagtgaggacaaagtcatagaaaaatagttaagtatgaaatgagttatctattgatttattatgataaggactgtgcctgacacaatggtttaaggaagagacaggaaaactcaatttctactctcgatttcctgtaaaatcagtgacaaagaattcttagattatttcaaacttcccttagatactgagctcagtaaattgttctaggaaattatctctcatttcagactttctcacatgagacatgttaccatcttttggctttctgactatcgaaaaaaatagataaaatttccataaacagaagaattataccaccactgttcaataattgcctttaaaatatttcacatttcatttaaaagttctcttcaaccttgtgataaaatggtcaagaatttttctaatagtaaagttccaacaattttgttatgccgagttgctcagttgtgtctgactcttgtgactccatggactgtagcccaccaggctcttctgtccatggggattctccaggcaagaatactggagtgggttgccatgccctcctccaggggatatttccaaccaagggatcaaacccaggtctccctcattgtaggcagattcttaattgtctgacctaccagggaaaccctccaacaattttagtcaaattcaaaatatcccttaatgctaaccttaactgtatatccaaagtttctcatttccaaattatctagaagcagtcctaagccaaaaaacaggtgttatgctctgaatggtattatttatactaatggaataaattgtagtgttaagttttgctattaattttatatcagcactgaataacttctttgaaattttctgacttagtctaaaccaattagaaagtgtaaaatctcattctcagctctagagcaagaaagtaaacacataaatttattcagcattttcaagtcaattataaatatataagatacccaccaatatcttctccaggctctgacaggcctcctgggaacttccacatgtttttcagctgtagtattaaatcagaaagcaaagttaacacagctcttatttactaacatacacatacgtagagatgccacagaagctacccataattgatcaaggtggttgagaatttattttttcgtaactgccaccaatttttttcagcttccttcctcactcctttcttctctcgggaaactgctgacttgtgaaatctttcctatctttttatttaggaaatagaagtggttttttttatgttaatgtgataaattctgtatgagtgaaacagtggggggaacatctactgaatttgtatagttaaaaatttttgctgctagtttattaaagaatacatgaatcttactgatgctgctataaattagtagaaaatatataaatgtaatcactaaagtatgctatttttaattttcaatttactttctatattgtgtgtctaatcagatatattaatcttaagagttttcttgttctctgtgttaatgattttatgtaaaaatataattgtctttcctgggaagtgtgaataaaattgatttaagtttctggct

(19) United States
(12) Patent Application Publication
(10) Pub. No.: US 2023/0220332 A1
(43) Pub. Date: Jul. 13, 2023

(54) MAMMALIAN LIVESTOCK PLURIPOTENT STEM CELLS FROM DELAYED EMBRYOS

(71) Applicant: Accellta Ltd., Haifa (IL)

(72) Inventor: Michal AMIT, Yuvalim (IL)

(73) Assignee: Accellta Ltd., Haifa (IL)

(21) Appl. No.: 18/092,495

(22) Filed: Jan. 3, 2023

Related U.S. Application Data
(63) Continuation of application No. PCT/IL2021/050817, filed on Jul. 1, 2021.
(60) Provisional application No. 63/047,375, filed on Jul. 2, 2020.

(51) Int. Cl. C12N 5/073; C12N 5/077

(52) U.S. Cl. CPC C12N 5/0604; C12N 5/0653; C12N 2501/115; C12N 2506/02

(57) ABSTRACT

Provided are methods of deriving a mammalian livestock pluripotent stem cells line, by (a) ex-vivo culturing a mammalian livestock embryo of at least 7 days post-fertilization for a culturing period of at least 4 days and no more than until 21 days post-fertilization so at to obtain an embryo comprising epiblast cell and/or late stage pluripotent stem cell; (b) isolating from the embryo the epiblast cell and/or the late stage pluripotent stem cell, and (c) culturing the epiblast cell and/or the late-stage pluripotent stem cell under conditions suitable for expansion of undifferentiated mammalian livestock pluripotent stem cells to thereby obtain a population of mammalian livestock pluripotent stem cells. Also provided are isolated mammalian livestock pluripotent stem cells, and cells differentiated therefrom.

### RELATED APPLICATIONS

This application is a Continuation of PCT Patent Application No. PCT/IL2021/050817 having International filing date of Jul. 1, 2021, which claims the benefit of priority under 35 USC § 119(e) of U.S. Provisional Patent Application No. 63/047,375 filed on Jul. 2, 2020. The contents of the above applications are all incorporated by reference as if fully set forth herein in their entirety.

### SEQUENCE LISTING STATEMENT

The XML file, entitled 95140SequenceListing.xml, created on Jan. 3, 2023, comprising 31,505 bytes, submitted concurrently with the filing of this application is incorporated herein by reference.

### FIELD AND BACKGROUND OF THE INVENTION

The present invention, in some embodiments thereof, relates to an isolated mammalian livestock pluripotent stem cell and methods of generating same, and, more particularly, but not exclusively, to a mammalian livestock (e.g., bovine) pluripotent stem cells cultures and cells differentiated therefrom.

Embryonic development starts soon after fertilization with blastomer cleavage, proliferation and differentiation. The blastomers within the developing mammalian embryo remain totipotent until the morula compaction stage. In the compacted embryo the blastomers initiate polarization which results in two distinct cell-populations; the inner cell mass (ICM), which will contribute to the embryo proper, and the outer trophoectoderm layer, which develops into the extra embryonic layers. Soon after implantation is acquired, the ICM is separated into a layer of primitive endoderm, which gives rise to the extra embryonic endoderm, and a layer of primitive ectoderm, which gives rise to the embryo proper and to some extra embryonic derivatives [Gardner 1982]. After implantation and gastrulation, the cells become progressively restricted to a specific lineage, thus their pluripotency is lost and they are regarded as multi-potent progenitor cells. Therefore, it should be noted that pluripotent embryonic stem cells proliferate and replicate in the intact embryo only for a limited period of time.

Embryonic stem cell (ESC) lines are pluripotent lines derived from the mammalian embryo at the blastocyst stage. Though human ESCs had been isolated and characterized [Thomson et al. 1998: Reubinoff et al. 2000], the pluripotency of non-cultured human post-implantation embryonic cells between the time of implantation and the gastrulation process has never before been examined.

The ability to culture human embryos in vitro to day 9 had been previously reported, demonstrating proliferating and healthy ICM [Edwards and Surani, 1978], yet these reports did not provide answers to a few critical questions, such as whether pluripotent stem cells still exist in the post-implantation embryo and whether it is feasible to isolate and culture them continuously to allow their characterization.

WO2006/040763 discloses isolated primate embryonic cells characterized by expression of brachyury and the ability to differentiate to derivatives of each of an endoderm, mesoderm, and ectoderm tissue. The isolated cells were generated by culturing human blastocysts on MEFs as whole embryos for 9-14 days post fertilization until a large cyst was developed.

Derivation of bovine embryonic stem cells (ESCs) had been reported to have low success rates (Mitalipova et al, 2001), and only few studied reported the derivation of characterized bovine ESCs.

Recently, Bogliotti Y. S., et al., 2018 (PNAS, 115: 2090-2095) describe the derivation of stable primed pluripotent embryonic stem cells from bovine blastocysts using a TeSR1-base medium supplemented with FGF2 and a WNT signaling pathway inhibitor (IWR1), with a derivation efficiency of 44-58%. The resulting bovine ESCs exhibited a SOX2⁺/OCT4⁺/CDX2⁻/GATA6⁻ expression signature, but did not exhibit the clearly defined colony margins that are characteristics to human ESCs and mouse EpiSCs.

However, there is no former report on the derivation of epiblast stage or late stage embryo PSCs from mammalian livestock such as bovines.

### SUMMARY OF THE INVENTION

According to an aspect of some embodiments of the present invention there is provided a method of deriving a mammalian livestock pluripotent stem cells line, the method comprising:

(a) ex-vivo culturing a mammalian livestock embryo of at least 7 days post-fertilization for a culturing period of at least 4 days and no more than until 21 days post-fertilization so at to obtain an embryo comprising an epiblast cell and/or a late stage pluripotent stem cell;

(b) isolating from the embryo the epiblast cell and/or the late stage pluripotent stem cell, and

(c) culturing the epiblast cell and/or the late-stage pluripotent stem cell under conditions suitable for expansion of undifferentiated mammalian livestock pluripotent stem cells to thereby obtain a population of mammalian livestock pluripotent stem cells,

thereby deriving the mammalian livestock pluripotent stem cells line.

According to an aspect of some embodiments of the present invention there is provided an isolated mammalian livestock pluripotent stem cell generated by the method of some embodiments of the invention, wherein the isolated mammalian livestock pluripotent stem cell is capable of differentiating into the ectoderm, mesoderm and ectoderm embryonic germ layers, and is capable of spontaneous differentiation into adipogenic cells when cultured in a medium devoid of dexamethasone.

According to an aspect of some embodiments of the present invention there is provided a method of generating an adipocyte, comprising culturing the isolated mammalian livestock pluripotent stem cell of some embodiments of the invention, or the population of mammalian livestock pluripotent stem cells obtained by the method of some embodiments of the invention in a culture medium devoid of chemical or hormonal induction towards adipogenic lineage for at least 10 days and no more than 60 days without passaging, thereby generating the adipocyte.

According to an aspect of some embodiments of the present invention there is provided a method of preparing food product, comprising incorporating the adipocyte generated by the method of some embodiments of the invention with a food product, thereby preparing the food product.

According to an aspect of some embodiments of the present invention there is provided a food product comprising the adipocyte generated by the method of some embodiments of the invention.

According to some embodiments of the invention, the mammalian livestock pluripotent stem cells are capable of spontaneous differentiation into adipocytes in an absence of adipogenic differentiation agent(s).

According to some embodiments of the invention, the mammalian livestock pluripotent stem cells are capable of spontaneous differentiation into adipocytes when cultured in a medium devoid of dexamethasone.

According to some embodiments of the invention, isolating is effected when the embryo has developed a cyst characterized by a diameter of about 0.4 millimeter (mm) to about 1 mm.

According to some embodiments of the invention, the epiblast cell and/or the late-stage pluripotent stem cell are comprised in a disc-like structure in the embryo, and wherein the isolating further comprises removal of trophoectoderm cells or cells differentiated from the trophoectoderm cells surrounding the disc-like structure.

According to some embodiments of the invention, the method further comprising removing a zona pellucida of the embryo prior to culturing the mammalian livestock embryo.

According to some embodiments of the invention, culturing the mammalian livestock embryo further comprising re-plating the mammalian livestock embryo on a fresh feeder cell layer or fresh extracellular matrix during the culturing period.

According to some embodiments of the invention, the method further comprising removing surrounding fibroblasts from the mammalian livestock embryo prior to the re-plating.

According to some embodiments of the invention, the epiblast cell and/or the late-stage pluripotent stem cell are characterized by a large nucleus to cytoplasm ratio.

According to some embodiments of the invention, the method further comprising mechanically passaging the population of mammalian livestock pluripotent stem cells for at least 2 passages to thereby obtain a population enriched with the mammalian livestock pluripotent stem cells.

According to some embodiments of the invention, the method further comprising mechanically passaging the population of mammalian livestock pluripotent stem cells for about 4-6 passages to thereby obtain a population enriched with the mammalian livestock pluripotent stem cells.

According to some embodiments of the invention, passaging the population enriched with the mammalian livestock pluripotent stem cells is performed every 5-10 days.

According to some embodiments of the invention, passaging the population enriched with the mammalian livestock pluripotent stem cells is performed by enzymatic passaging.

According to some embodiments of the invention, passaging the population enriched with the mammalian livestock pluripotent stem cells is performed by mechanical passaging.

According to some embodiments of the invention, culturing the mammalian livestock embryo is performed on a two-dimensional culture system.

According to some embodiments of the invention, culturing the mammalian livestock embryo is performed on feeder cells.

According to some embodiments of the invention, culturing the epiblast cell and/or the late-stage pluripotent stem cell is performed on a two-dimensional culture system.

According to some embodiments of the invention, the two-dimensional culture system comprises a feeder-free matrix.

According to some embodiments of the invention, the feeder-free matrix is selected from the group consisting of a Matrigel™ matrix, a fibronectin matrix, a laminin matrix, and a vitronectin matrix.

According to some embodiments of the invention, isolating the epiblast cell and/or the late stage pluripotent stem cell is effected using syringe needle under stereoscope.

According to some embodiments of the invention, culturing the mammalian livestock embryo is performed in a culture medium comprising a defined fetal bovine serum.

According to some embodiments of the invention, the culture medium comprises a base medium selected from the group consisting of DMEM\F12, KO-DMEM and DMEM.

According to some embodiments of the invention, culturing the mammalian livestock embryo is performed in a culture medium comprising the IL6RIL6 chimera.

According to some embodiments of the invention, culturing the epiblast cell and/or the late stage pluripotent stem cell is performed in a culture medium comprising the IL6RIL6 chimera.

According to some embodiments of the invention, the culture medium further comprises basic fibroblast growth factor (bFGF).

According to some embodiments of the invention, the culture medium further comprises serum replacement.

According to some embodiments of the invention, culturing the mammalian livestock embryo is performed in a culture medium comprising a Wnt3a polypeptide.

According to some embodiments of the invention, culturing the epiblast cell and/or the late stage pluripotent stem cell is performed in a culture medium comprising a Wnt3a polypeptide.

According to some embodiments of the invention, the culture medium further comprising basic fibroblast growth factor (bFGF) and leukemia inhibitory factor (LIF).

According to some embodiments of the invention, the culture medium further comprising serum replacement.

According to some embodiments of the invention, the mammalian livestock embryo is obtained from in vitro fertilization of a mammalian livestock oocyte.

According to some embodiments of the invention, the mammalian livestock embryo is obtained by Nuclear Transfer (NT) of mammalian livestock cell.

According to some embodiments of the invention, the mammalian livestock embryo is obtained by parthenogenesis.

According to some embodiments of the invention, the bovine embryo is obtained from in vitro fertilization of a bovine oocyte.

According to some embodiments of the invention, the bovine embryo is obtained by Nuclear Transfer (NT) of bovine cell.

According to some embodiments of the invention, the bovine embryo is obtained by parthenogenesis.

According to some embodiments of the invention, the mammalian livestock e embryo is placed on the two-dimensional culture system using a 27 g needle or a pulled Pasteur Pipette.

According to some embodiments of the invention, the mammalian livestock embryo is placed on the feeder cells using a 27 g needle or a pulled Pasteur Pipette.

According to some embodiments of the invention, prior to the culturing the mammalian livestock embryo is covered with a drop of an extracellular matrix.

According to some embodiments of the invention, the cells of the population of mammalian livestock pluripotent stem cells are capable of differentiation into the endoderm, mesoderm and ectoderm embryonic germ layers.

According to some embodiments of the invention, the cells of the population of mammalian livestock pluripotent stem cells are capable of differentiation into embryoid bodies.

According to some embodiments of the invention, the cells of the population of mammalian livestock pluripotent stem cells spontaneously differentiate into adipogenic cell lineage when cultured without passaging for about 14-21 days in a culture medium.

According to some embodiments of the invention, the culture medium comprises serum.

According to some embodiments of the invention, the culture medium comprises the IL6RIL6 chimera.

According to some embodiments of the invention, the culture medium is devoid of dexamethasone.

According to some embodiments of the invention, the culture medium comprises serum.

According to some embodiments of the invention, the culture medium comprises the IL6RIL6 chimera.

According to some embodiments of the invention, the mammalian livestock is a ruminant mammalian livestock.

According to some embodiments of the invention, the mammalian livestock is a non-ruminant mammalian livestock.

According to some embodiments of the invention, the ruminant mammalian livestock is selected from the group consisting of a Bovinae subfamily, sheep, goat, deer, and camel.

According to some embodiments of the invention, the ruminant mammalian livestock of the Bovinae subfamily is a cattle or a yak.

According to some embodiments of the invention, the ruminant mammalian livestock of the Bovinae subfamily is a cattle.

According to some embodiments of the invention, the cattle is buffalo, bison or cow (bovine).

According to some embodiments of the invention, the mammalian livestock is cow (bovine).

According to some embodiments of the invention, the cattle is cow (bovine).

According to some embodiments of the invention, the non-ruminant mammalian livestock is selected from the group pig, rabbit, and horse.

According to some embodiments of the invention, the mammalian livestock is horse.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C are images depicting derivation of a bovine pluripotent stem cell lines (bPSC line) from delayed blastocysts. FIG. 1A—Bovine blastocytes with notable inner cell mass (ICM), 8 days post insemination. FIG. 1B—Whole embryo plated with mouse embryonic fibroblasts (MEFs), 11 days post insemination. A notable cyst is developed. FIG. 1C—The same embryo, 14 days post insemination. The cyst is further grown, and a secondary cyst was developed. Size bars: FIG. 1A—1 mm (millimeter); FIG. 1B—1 mm; FIG. 1C—1 mm.

FIGS. 2A-D are images depicting the morphology of bPSC colonies cultured under different culture conditions. FIG. 2A—bPSC colony, cultured on MEFs in the presence of a culture medium (“medium X”) which includes serum. FIG. 2B—bPSC colony, cultured on Matrigel™ matrix in the presence of a serum-free culture medium (the IL6RIL6 Chimera). FIG. 2C—bPSC colony, cultured on MEFs in the presence of a culture medium supplemented with serum-replacement (the IL6RIL6 Chimera). FIG. 2D—enlarged image of the image shown in FIG. 2A. In FIG. 2A (and more clearly in the enlarged FIG. 2D) and in FIG. 2C it is noted that there are spaces between the cells within the colony, and the cells have a high nucleus to cytoplasm ratio, which is a typical characteristics of pluripotent stem cells (PSC). Size bars: FIG. 2A—1 mm; FIG. 2B—1 mm; FIG. 2C—1 mm; FIG. 2D—1 mm.

FIGS. 3A-B are images depicting immunofluorescence staining of a key pluripotency marker OCT4. FIG. 3A—DAPI staining of the same field as in FIG. 3B. FIG. 3B—Positive staining of Oct4 (red). Size bars: FIG. 3A—100 μm (micrometer); FIG. 3B—100 μm.

FIGS. 4A-C are images depicting the morphology of bPSC, at passage 3, that spontaneously differentiated while cultured in the presence of a culture medium such as DMEM enriched with 10-20% v/v of FBS. FIGS. 4A and 4B depict examples for bPSC colonies consisting differentiating cells. FIG. 4C—Cystic EB formed by bPSC cultured in a culture medium (“Medium X”) which includes serum. Size bars: FIG. 4A—100 μm; FIG. 4B—50 μm; FIG. 4C—100 μm.

FIGS. 5A-D are images depicting immunofluorescence staining of key differentiation markers following spontaneous differentiation of bPSCs in culture, demonstrating representative cells of the three embryonic germ layers. FIG. 5A—Positive staining of alfa-Fetoprotein (representative of the endoderm germ layer); FIG. 5B—The same staining of FIG. 5A) merged with DAPI (nuclear) staining. FIG. 5C-D—FIG. 5D shows co-staining of EMOS (red, representative of the mesoderm germ layer) and 3-beta-tubulin (green, representative of the ectoderm germ layer) with a DAPI (blue, nuclear staining). FIG. 5 C shows only the DAPI nuclear staining of the same microscopic field shown in FIG. 5D. Size bars: FIG. 5A—100 m; FIG. 5B—100 μm; FIG. 5C—50 μm; FIG. 5D—50 μm.

FIGS. 6A-B are images depicting spontaneous differentiation of bovine pluripotent cells into adipocytes (fat cells). The bovine PSCs were cultured in a medium supplemented with serum (Medium X) without being passaged for at least 14 days, following which the cells underwent a spontaneous differentiation into adipocytes exhibiting lipid droplets. Lipid droplets within the cells (white arrows) in FIGS. 6A-B were positively stained by Oil Red staining. Size bars: FIG. 6A—20 μm; FIG. 6B—50 μm;

FIGS. 7A-B are images depicting derivation of bovine pluripotent stem cells (bPSC) line BVN6 from delayed bovine blastocysts. FIG. 7A—Bovine blastocytes, 8 days post insemination; FIG. 7B—Whole embryo plated with MEFs, 16 days post insemination. A notable cyst is developed (white arrow in FIG. 7B). Culture medium used for derivation of the bovine delayed blastocyst cell line is medium X. Scale Bars: FIG. 7A: 50 μm; FIG. 7B: 200 μm.

FIGS. 8A-D are images depicting the derivation of horse PSC line from delayed blastocysts. FIG. 8A—Horse extended blastocysts with notable inner cell mass (ICM; white arrow), 8 days post insemination. FIG. 8B—Whole horse embryo plated with mouse embryonic fibroblasts (MEFs), 16 days post insemination. A notable cyst is developed (arrow). The microscopic focus is on the cyst. FIG. 8C—The same field as in FIG. 8B, while the microscopic focus is on the cells. FIG. 8D—Derived cells colony at passage 2 grown with medium X. Scale Bars: FIG. 8A—200 μm; FIG. 8B—100 μm; FIG. 8C—100 μm; and FIG. 8D—50 μm.

FIGS. 9A-D are images depicting the morphology of a bovine pluripotent stem cell (bPSCs) colony. FIG. 9A—bPSC line BVN1 at passage 30 (p30), an overall look of a colony; FIG. 9B—bPSC line BVN1 at p30, a higher magnification to enable seeing the cell nuclei in the cells of the colony; FIG. 9C—bPSC line BVN2 at p8; FIG. 9D—bPSC line BVN5 at p9. All cell lines were derived using medium X. Scale Bars: FIG. 9A—100 μm; FIG. 9B—50 μm; FIG. 9C—50 μm; FIG. 9D—100 μm.

FIGS. 10A-D are images depicting immunofluorescence staining of key pluripotency markers TRA1-60 (red) and TRA1-81 (green) in the bPSC line BVN5 at passage 8 (p8). FIG. 10A—DAPI (nuclear counterstaining) of the cells shown in FIG. 10B; FIG. 10B—Positive staining of TRA1-60; FIG. 10C—DAPI of the cells shown in FIG. 10D; FIG. 10D—Positive staining of TRA 1-81. Scale Bars: FIG. 10A—50 μm; FIG. 10B—50 μm; FIG. 10C—100 μm; FIG. 10D—100 μm.

### DESCRIPTION OF SPECIFIC EMBODIMENTS OF THE INVENTION

The present invention, in some embodiments thereof, relates to an isolated mammalian livestock pluripotent stem cell and methods of generating same, and, more particularly, but not exclusively, to a mammalian livestock (e.g., bovine, horse) pluripotent stem cells cultures and cells differentiated therefrom.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Derivation of bovine embryonic stem cells had been reported to have low success rates (Mitalipova et al, 2001), and only a few studies reported the derivation of characterized bovine ESCs.

Recently, Bogliotti Y. S., et al., 2018 (PNAS, 115: 2090-2095) describe the derivation of stable primed pluripotent embryonic stem cells from bovine blastocysts using a TeSR1-base medium supplemented with FGF2 and a WNT signaling pathway inhibitor (IWR1), with a derivation efficiency of 44-58%. The resulting bovine ESCs exhibited a SOX2⁺/OCT4+/CDX2⁻/GATA6⁻ expression signature, but did not exhibit the clearly defined colony margins that are characteristics to human ESCs and mouse EpiSCs (Epiblast stem cells).

However, there is no former report on the derivation of epiblast stage or late stage embryo PSCs from mammalian livestock such as bovines or horses.

The present inventor has surprisingly uncovered that mammalian livestock pluripotent stem cells (bPSCs) can be isolated from a mammalian livestock embryo (such as a bovine or a horse embryo) which is cultured ex-vivo beyond the blastocyst stage (day 7 post-fertilization) for at least 4 days and no more than until 21 days post-fertilization. Example 1 of the Examples section which follows shows derivation of several bovine pluripotent stem cells lines from various bovine embryos of 7 days post-insemination, which is considered to be no more than 7 days post-fertilization (in-vivo). Usually fertilization occurs in-vivo (in uterus) within 0-24 hours post insemination of the mammalian livestock female (e.g., cow or horse). The 7-day old post-insemination embryos are at an early blastocyst or blastocyst stage. The embryos were removed by washing from the cow's uterus, and were then cultured ex-vivo for 6-13 days (thus being 13-20 day-old embryos post insemination). The 13-20 day-old post insemination embryos were observed under the microscope and were evaluated for the formation of a disc-like structure, comprising epiblast and late stage pluripotent stem cells. The disc-like structure was removed from each embryo and the isolated cells comprised in the disc-like structure were cultured in-vitro while being serially passaged every 4-10 days, thus obtaining a population of bovine pluripotent stem cells. The bovine pluripotent stem cell lines were termed “BVN1”, “BVN2”, “BVN5” and “BVN6”. It is noted that the embryo of BVN1 was cultured ex-vivo for 7 days post insemination; the embryo of BVN2 was cultured ex-vivo for 12 days post insemination; the embryo of BVN5 was cultured ex-vivo for 13 days post insemination; and the embryo of BVN6 was cultured ex-vivo for 11 days post insemination, following which the disc-like structures (which comprise the epiblast and late stage pluripotent stem cells) were removed and the epiblast and late stage pluripotent stem cells were cultured in-vitro while being passaged serially every 4-10 days.

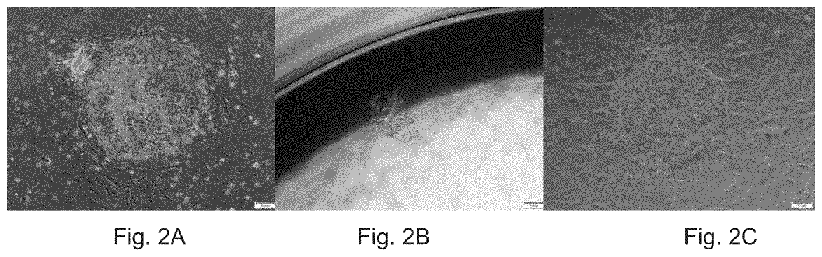

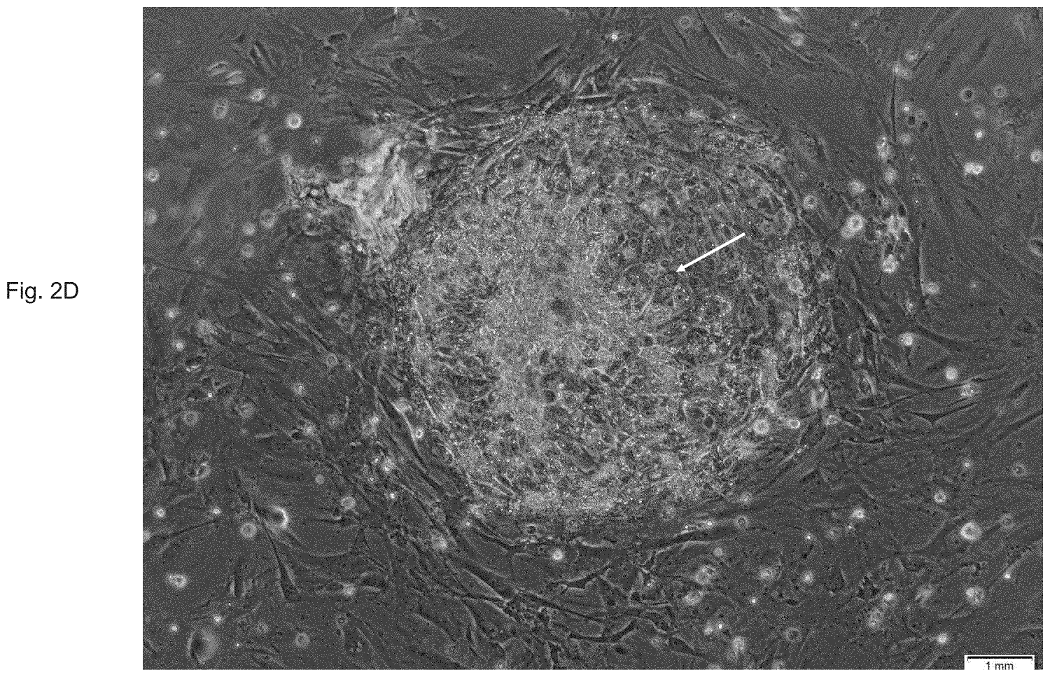

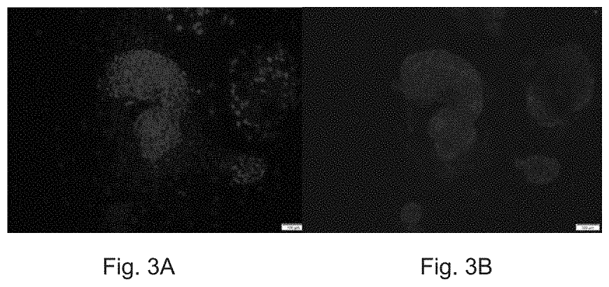

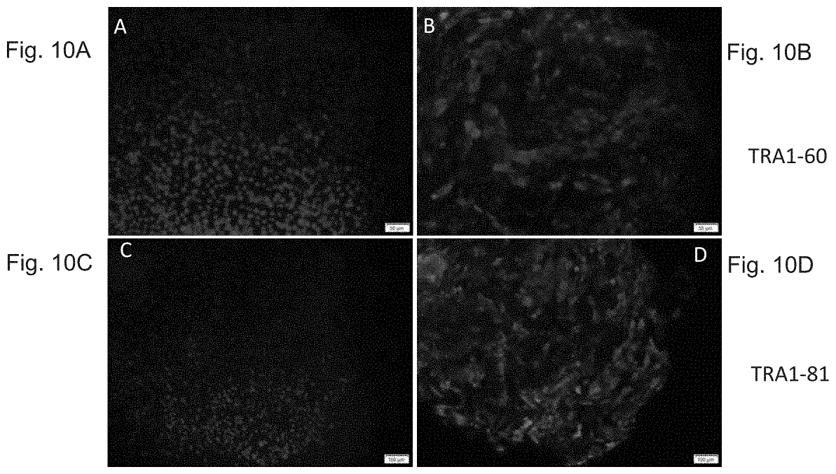

The Examples section which follows shows that the bovine PSCs were cultured on feeder cell layers (FIGS. 2A, 2C and 2D) or on a matrix (e.g., Matrigel™, FIG. 2B) while maintaining their pluripotency as is evidenced by the expression of OCT4 (FIGS. 3A-B), TRA1-60 and TRA1-81 (FIG. 10A-D).

Example 2 of the Examples section which follows shows derivation of a horse pluripotent stem cells line from a horse (mare) embryo of 8 days post-insemination which was cultured ex-vivo for 8 days (thus being a 16-day old post insemination embryo), following which a disc-like structure, comprising epiblast and late stage pluripotent stem cells, was removed from the embryo and the isolated cells were cultured in-vitro while being serially passaged every 5-10 days, thus obtaining a population of horse pluripotent stem cells. The horse pluripotent stem cell lines was termed “HRS1”.

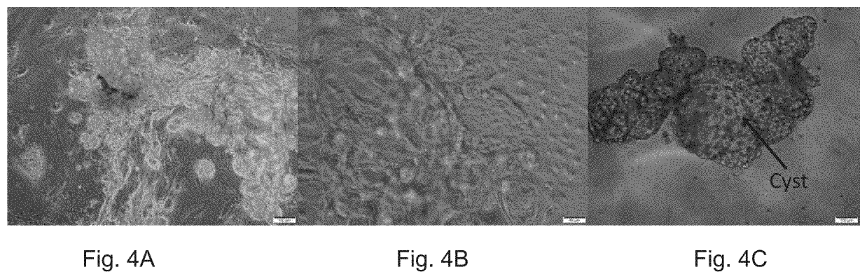

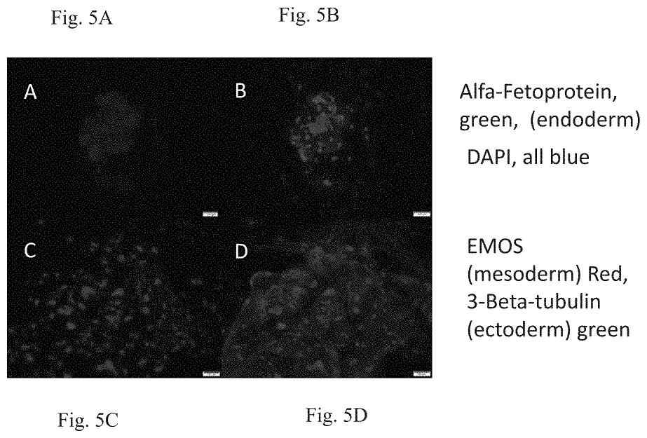

The Examples section which follows further shows that upon removal from their feeder cell layers or their supporting matrix, and in the presence of a serum-containing medium (e.g., “Medium X”), the bPSCs (bovine pluripotent stem cells) spontaneously differentiated into embryoid bodies (FIGS. 4A-C) containing differentiated cells from all three embryonic germ layers, i.e., mesoderm, ectoderm and endoderm (FIGS. 5A-D).

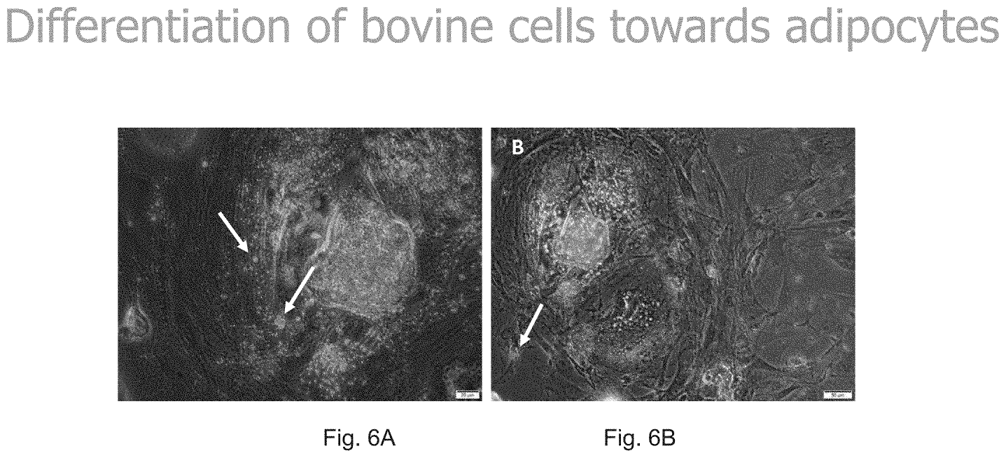

In addition, when the bPSCs were left without passaging for 14-21 days on a 2-dimensional culture system in a medium devoid of any adipogenic-differentiation agents (such as dexamethasone) the cells spontaneously differentiated into adipocytes that are positively stained with the Oil red staining (FIGS. 6A-B).

According to an aspect of some embodiments of the invention, there is provided a method of deriving a mammalian livestock pluripotent stem cells line, the method comprising:

(a) ex-vivo culturing a mammalian livestock embryo of at least 7 days post-fertilization for a culturing period of at least 4 days and no more than until 21 days post-fertilization so at to obtain an embryo comprising an epiblast cell and/or a late stage pluripotent stem cell,
(b) isolating from the embryo the epiblast cell and/or the late stage pluripotent stem cell, and
(c) culturing the epiblast cell and/or the late-stage pluripotent stem cell under conditions suitable for expansion of undifferentiated mammalian livestock pluripotent stem cells to thereby obtain a population of mammalian livestock pluripotent stem cells,
thereby deriving the mammalian livestock pluripotent stem cells line.

As used herein, the phrase “stem cells” refers to cells which are capable of remaining in an undifferentiated state (e.g., totipotent, pluripotent or multipotent stem cells) for extended periods of time in culture until induced to differentiate into other cell types having a particular, specialized function (e.g., fully differentiated cells).

The phrase “pluripotent stem cells” refers to cells which can differentiate into all three embryonic germ layers, i.e., ectoderm, endoderm and mesoderm, or remaining in an undifferentiated state.

As used herein the phrase “deriving” with respect to “a mammalian livestock pluripotent stem cells line” refers to generating a population of mammalian livestock pluripotent stem cells from at least one stem cell (e.g., an epiblast cell or a late-stage pluripotent stem cell) that is isolated from a single mammalian livestock embryo (e.g., from an ex-vivo cultured bovine embryo).

According to the method of some embodiments of the invention, a mammalian livestock embryo of at least 7 days post-fertilization is cultured ex-vivo. It should be noted that at 7 days post-fertilization the mammalian livestock embryo is at the blastocyst stage, characterized by the existence of an inner cell mass (ICM), a trophoblast layer and a cyst.

According to some embodiments of the invention, the mammalian livestock embryo is obtained before implantation of the embryo in the uterus.

According to some embodiments of the invention, the mammalian livestock embryo is obtained from in vitro fertilization of a mammalian livestock oocyte.

According to some embodiments of the invention, the mammalian livestock embryo is obtained by Nuclear Transfer (NT) of a mammalian livestock cell. Methods of nuclear transfer are known in the art and described for example in Steven L. Stice., et al., 1996 (“Pluripotent Bovine Embryonic Cell Lines Direct Embryonic Development Following Nuclear Transfer”; BIOLOGY OF REPRODUCTION 54, 100-110), which is fully incorporated herein by reference in its entirety, and include, for example, nuclear transfer to oocytes, or nuclear transfer into zygotes if the recipient cells are arrested in mitosis.

According to some embodiments of the invention, the mammalian livestock embryo is obtained by parthenogenesis, e.g., by stimulating an unfertilized ova (parthenotes) as described for example in Kitai Kim et al., 2007 (“Histocompatible Embryonic Stem Cells by Parthenogenesis”; SCIENCE, VOL 315; pages: 482-486), which is fully incorporated herein by reference in its entirety.

According to some embodiments of the invention, the mammalian livestock embryo is placed on a two-dimensional culture system or on a feeder cell layer using a 27 g needle or a pulled Pasteur Pipette.

According to some embodiments of the invention, prior to culturing ex-vivo the mammalian livestock embryo is covered with a drop of an extracellular matrix.

The extracellular matrix can be composed of components derived from basement membrane and/or extracellular matrix components that form part of adhesion molecule receptor-ligand couplings. MATRIGEL® (Becton Dickinson, USA) is one example of a commercially available matrix which is suitable for use with the present invention. MATRIGEL® is a soluble preparation from Engelbreth-Holm-Swarm tumor cells that gels at room temperature to form a reconstituted basement membrane; MATRIGEL® is also available as a growth factor reduced preparation. Other extracellular matrix components and component mixtures which are suitable for use with the present invention include foreskin matrix, laminin matrix, fibronectin matrix, proteoglycan matrix, entactin matrix, heparan sulfate matrix, collagen matrix and the like, alone or in various combinations thereof.

According to some embodiments of the invention the matrix is xeno-free.

The term “xeno” is a prefix based on the Greek word “Xenos”, i.e., a stranger. As used herein the phrase “xeno-free” refers to being devoid of any components which are derived from a xenos (i.e., not the same, a foreigner) species. Such components can be contaminants such as pathogens associated with (e.g., infecting) the xeno species, cellular components of the xeno species or a-cellular components (e.g., fluid) of the xeno species.

In cases where complete xeno-free culturing conditions are desired, the matrix is preferably derived from the same source of the embryo, e.g., a mammalian livestock, e.g., a bovine, or can be synthesized using recombinant techniques. Such matrices include, for example, recombinant fibronectin, recombinant laminin, a synthetic fibronectin matrix, Vitronectin matrix, and/or a collagen matrix. A synthetic fibronectin matrix can be obtained from Sigma, St. Louis, Mo., USA.

According to some embodiments of the invention, the method further comprising removing the zona pellucida of the mammalian livestock embryo prior to culturing the mammalian livestock embryo ex-vivo.

Methods of removing the zona pellucida include, but are not limited to chemical digestion (e.g., with a Tyrode's acidic solution), enzymatic digestion (e.g., using a trypsin-like enzyme or Collagenase), or a mechanical methods using e.g., micropipettes, or a micromanipulator (e.g., using a laser).

According to some embodiments of the invention, the zona pellucida is removed by chemical digestion with a Tyrode's acidic solution.

According to some embodiments of the invention, ex-vivo culturing the mammalian livestock embryo is performed on a two-dimensional culture system.

According to some embodiments of the invention, ex-vivo culturing the mammalian livestock embryo is performed on feeder cells.

Once placed on the two-dimensional culture system or on the feeder cell layer, the mammalian livestock embryo spontaneously attaches to the surface of the two-dimensional culture system or to the feeder cell layers and continues to grow and develop ex-vivo.

According to the method of some embodiments of the invention, the mammalian livestock embryo is cultured ex-vivo under conditions which enable its further development outside the mammalian livestock uterus, so as to obtain an embryo comprising epiblast cell and/or late stage pluripotent stem cell.

According to some embodiments of the invention, the conditions which enable the development of the mammalian livestock embryo outside the mammalian livestock uterus include a culturing system (e.g., a feeder cell layer or a matrix) and a suitable culture medium, which enables the undifferentiated growth of epiblast cells and late-stage pluripotent stem cells that are contained within the mammalian livestock embryo.

As mentioned, the method according to some embodiments of the invention comprising ex-vivo culturing of a mammalian livestock embryo for at least 4 days in a culture medium.

According to some embodiments of the invention, the culture medium used for ex-vivo culturing the mammalian livestock embryo comprises a base medium and serum.

According to some embodiments of the invention, ex-vivo culturing the mammalian livestock embryo is performed in a culture medium comprising a defined fetal bovine serum.

According to some embodiments of the invention, the culture medium comprises a base medium selected from the group consisting of DMEM\F12 and KO-DMEM and DMEM.

According to some embodiments of the invention, the culture medium used for ex-vivo culturing the mammalian livestock embryo is serum-free.

As used herein the phrase “serum-free” refers to being devoid of a human or an animal serum.

It should be noted that the function of serum in culturing protocols is to provide the cultured cells with an environment similar to that present in vivo (i.e., within the organism from which the cells are derived, e.g., a blastocyst of an embryo). However, the use of serum which is derived from either an animal source (e.g., mammalian livestock, e.g., bovine serum) or a human source (human serum) is limited by the significant variations in serum components between individuals and the risk of having xeno contaminants (in case of a serum from another species used).

According to some embodiments of the invention, the serum-free culture medium does not comprise serum or portions thereof.

According to some embodiments of the invention, ex-vivo culturing the mammalian livestock embryo is performed in a culture medium comprising the IL6RIL6 chimera.

According to some embodiments of the invention, the culture medium for ex-vivo culturing the mammalian livestock embryo, comprises the IL6RIL6 chimera at a concentration of about 100 pg/ml to about 300 pg/ml (e.g., about 100 pg/ml).

According to some embodiments of the invention, the culture medium for ex-vivo culturing the mammalian livestock embryo, comprises the IL6RIL6 chimera at a concentration of about 100 ng/ml to about 300 ng/ml (e.g., about 100 ng/ml).

According to some embodiments of the invention, the culture medium which comprises the IL6RIL6 chimera further comprises basic fibroblast growth factor (bFGF).

According to some embodiments of the invention, the culture medium for ex-vivo culturing the mammalian livestock embryo, which comprises the IL6RIL6 chimera, further comprises bFGF at a concentration of about 20 ng/ml to about 100 ng/ml (e.g., about 50 ng/ml, e.g., about 100 ng/ml).

According to some embodiments of the invention, the culture medium which comprises the IL6RIL6 chimera further comprises serum replacement.

According to some embodiments of the invention, the culture medium which comprises the IL6RIL6 chimera further comprises basic fibroblast growth factor (bFGF) and serum replacement.

According to some embodiments of the invention, ex-vivo culturing the mammalian livestock embryo is performed in a culture medium comprising a Wnt3a polypeptide.

According to some embodiments of the invention, the culture medium for ex-vivo culturing the mammalian livestock embryo, comprises the WNT3A polypeptide at a concentration from about 10 ng/ml to about 50 ng/ml (e.g., about 10 ng/ml).

According to some embodiments of the invention, the medium which comprises a Wnt3a polypeptide further comprises basic fibroblast growth factor (bFGF).

According to some embodiments of the invention, the culture medium for ex-vivo culturing the mammalian livestock embryo, which comprises the WNT3A polypeptide, further comprises bFGF at a concentration of about 20 ng/ml to about 100 ng/ml (e.g., about 50 ng/ml).

According to some embodiments of the invention, the medium which comprises a Wnt3a polypeptide further comprises leukemia inhibitory factor (LIF).

According to some embodiments of the invention, the culture medium for ex-vivo culturing the mammalian livestock embryo, which comprises the WNT3A polypeptide, further comprises LIF at a concentration from about 1000 U/ml (units per milliliter) to about 3000 U/ml (e.g., about 3000 U/ml).

According to some embodiments of the invention, the medium which comprises a Wnt3a polypeptide further comprises basic fibroblast growth factor (bFGF) and leukemia inhibitory factor (LIF).

According to some embodiments of the invention, the culture medium for ex-vivo culturing the mammalian livestock embryo, comprises Wnt3a polypeptide at a concentration range between 5-50 ng/ml, bFGF at a concentration range between 20-100 ng/ml, and LIF at a concentration range between 1000-3000 U/ml.

As used herein the phrase “culture medium” refers to a liquid substance used to support the growth of cells. The culture medium used by the invention according to some embodiments can be a water-based medium which includes a combination of substances such as salts, nutrients, minerals, vitamins, amino acids, nucleic acids, proteins such as cytokines, growth factors and hormones, all of which are needed for cell proliferation and/or differentiation.

For example, a culture medium according to an aspect of some embodiments of the invention can be a synthetic tissue culture medium comprising a basal medium such as the Dulbecco's Modified Eagle's Medium (DMEM, e.g., available for example from Gibco-Invitrogen Corporation products, Grand Island, N.Y., USA), DMEM/F12 (e.g., available for example from Biological Industries, Biet HaEmek, Israel), MEM alpha (e.g., available for example from Biological Industries, Biet HaEmek, Israel), Ham's F-12 (e.g., available for example from Invitrogen/Thermo Fisher Scientific), Ko-DMEM (e.g., available for example from Gibco-Invitrogen Corporation products, Grand Island, N.Y., USA), or Eagle's Minimum Essential Medium (EMEM, e.g., available for example from Gibco-Invitrogen Corporation products, Grand Island, N.Y., USA) supplemented with the necessary additives as is further described hereinunder. The concentration of the basal medium depends on the concentration of the other medium ingredients such as the serum replacement as discussed below.

According to some embodiments of the invention, the culture medium is a defined culture medium.

A “defined” culture medium refers to a chemically-defined culture medium manufactured from known components at specific concentrations. For example, a defined culture medium is a non-conditioned culture medium.

Conditioned medium is the growth medium of a monolayer cell culture (i.e., feeder cells) present following a certain culturing period. The conditioned medium includes growth factors and cytokines secreted by the monolayer cells in the culture.

Conditioned medium can be collected from a variety of cells forming monolayers in culture. Examples include mouse embryonic fibroblasts (MEF) conditioned medium, foreskin conditioned medium, human embryonic fibroblasts conditioned medium, human fallopian epithelial cells conditioned medium, and the like.

It should be noted that following a certain time in culture the feeder cells or matrix needs to be replaced with a fresh feeder cell layer or a fresh matrix of the same type in order to support the growth and development of the mammalian livestock embryo ex-vivo.

According to some embodiments of the invention, ex-vivo culturing the mammalian livestock (e.g., bovine) embryo further comprising re-plating the mammalian livestock embryo on a fresh feeder cell layer or fresh extracellular matrix during the culturing period.

According to some embodiments of the invention, the method further comprising removing surrounding fibroblasts from the mammalian livestock embryo prior to re-plating on the fresh feeder cell layers or matrix.

According to some embodiments of the invention, the ex-vivo culturing period of the mammalian livestock embryo is for least 4 days in culture, e.g., at least 5 days, at least 6 days, at least 7 days, at least 8 days, at least 9 days, at least 10 days, at least 11 days, at least 12 days, at least 13 days, and no more than until the embryo reaches 21 days post-fertilization.

According to some embodiments of the invention, the ex-vivo culturing period of the mammalian livestock embryo is for least 4 days in culture, e.g., at least 5 days, at least 6 days, at least 7 days, at least 8 days and no more than until the embryo reaches 21 days post-fertilization, no more than until the embryo reaches 20 days post-fertilization, no more than until the embryo reaches 19 days post-fertilization, no more than until the embryo reaches 18 days post-fertilization, no more than until the embryo reaches 17 days post-fertilization.

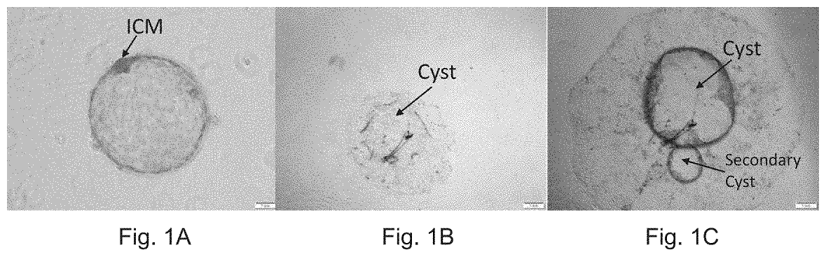

The present inventor has uncovered that when the mammalian livestock embryo that is cultured ex-vivo develops a cyst of a certain size (e.g., as shown in FIGS. 1B-C) the epiblast cells or the late-stage pluripotent stem cells contained in the embryo can be isolated and cultured in vitro for derivation of the pluripotent stem cell line.

According to some embodiments of the invention, when the cyst of the ex-vivo culture mammalian livestock embryo is characterized by a diameter of about 0.4 mm to about 1 mm then the epiblast cell or the late-stage pluripotent stem cells can be isolated and cultured in-vitro.

According to some embodiments of the invention, isolating is effected when the embryo has developed a cyst having a diameter of about 0.6-1 mm.

As used herein the phrase “epiblast cells” refers to cells of the embryonic epiblast. These cells are pluripotent and therefore capable of differentiating into all three embryonic germ layers.

As used herein the phrase “late stage pluripotent stem cells” refers to cells which are derived from the late epiblast stage until gastrulation. These cells are pluripotent and therefore capable of differentiating into all three embryonic germ layers.

According to some embodiments of the invention, the epiblast cell and/or the late-stage pluripotent stem cell are characterized by a large nucleus to cytoplasm ratio.

According to some embodiments of the invention, while inside the ex-vivo cultured mammalian livestock embryo, the epiblast cells or late-stage pluripotent stem cells are comprised in a disc-like structure.

Isolating the epiblast cells or late-stage pluripotent stem cells can be performed by removing the disc-like structure from the ex-vivo cultured embryo and transferring the cells contained in the disc-like structure into a fresh culture dish coated with a matrix or a feeder cell layer.

Isolating of epiblast cells or late-stage pluripotent stem cells from the ex-vivo cultured mammalian livestock embryo can be performed by various techniques, preferably using a microscope or a stereoscope.

For example, the epiblast cells or late-stage pluripotent stem cells can be captured using a syringe needle under stereoscope.

According to some embodiments of the invention, prior to culturing the cells of the disc-like structure on a culture dish (or culture vessel) the trophoectoderm cells or the cells differentiated from the trophoectoderm cells, which surround the disc-like structure, are removed.

The epiblast cells or late-stage pluripotent stem cells can then be cultured on either a feeder cell layer or on a matrix in a two-dimensional culture system in the presence of a suitable culture medium which maintains the cells in a pluripotent and undifferentiated state.

According to some embodiments of the invention, culturing the epiblast cell and/or the late-stage pluripotent stem cell is performed on a two-dimensional culture system.

According to some embodiments of the invention, the two-dimensional culture system comprises a feeder-free matrix.

As described above, once the epiblast cell and/or the late stage pluripotent stem cell is isolated from the mammalian livestock embryo these isolated cells are further cultured in vitro in the presence of a culture medium.

According to some embodiments of the invention, culturing the epiblast cell and/or the late stage pluripotent stem cell is performed in a culture medium comprising the IL6RIL6 chimera.

According to some embodiments of the invention, the culture medium for culturing the epiblast cell and/or the late stage mammalian livestock pluripotent stem cells comprises the IL6RIL6 chimera at a concentration range between 50-300 pg/ml (e.g., at concentration of about 100 pg/ml).

According to some embodiments of the invention, the culture medium for culturing the epiblast cell and/or the late stage mammalian livestock pluripotent stem cells comprises the IL6RIL6 chimera at a concentration range between 50-300 ng/ml (e.g., at concentration of about 100 ng/ml).

According to some embodiments of the invention, culturing the epiblast cell and/or the late stage pluripotent stem cell is performed in a culture medium comprising the IL6RIL6 chimera, basic fibroblast growth factor (bFGF) and serum replacement.

According to some embodiments of the invention, the culture medium for culturing the epiblast cell and/or the late stage mammalian livestock pluripotent stem cells comprises the IL6RIL6 chimera at a concentration range between 50-300 pg/ml (e.g., at concentration of about 100 pg/ml), bFGF at a concentration range between 20-100 ng/ml (e.g., at concentration of about 50 ng/ml) and serum replacement at a concentration range of 10-20% v/v (e.g., about 15% v/v).

According to some embodiments of the invention, the culture medium for culturing the epiblast cell and/or the late stage mammalian livestock pluripotent stem cells comprises the IL6RIL6 chimera at a concentration range between 50-300 ng/ml (e.g., at concentration of about 100 ng/ml), bFGF at a concentration range between 20-100 ng/ml (e.g., at concentration of about 50 ng/ml) and serum replacement at a concentration range of 10-20% v/v (e.g., about 15% v/v).

According to some embodiments of the invention, culturing the epiblast cell and/or the late stage pluripotent stem cell is performed in a culture medium comprising a Wnt3a polypeptide.

According to some embodiments of the invention, the culture medium for culturing the epiblast cell and/or the late stage mammalian livestock pluripotent stem cells comprises the Wnt3a polypeptide at a concentration range between 5-50 ng/ml (e.g., at concentration of about 10 ng/ml).

According to some embodiments of the invention, culturing the epiblast cell and/or the late stage pluripotent stem cell is performed in a culture medium comprising a Wnt3a polypeptide and basic fibroblast growth factor (bFGF).

According to some embodiments of the invention, the culture medium for culturing the epiblast cell and/or the late stage mammalian livestock pluripotent stem cells comprises the Wnt3a polypeptide at a concentration range between 5-50 ng/ml (e.g., at concentration of about 10 ng/ml), and bFGF at a concentration range between 20-100 ng/ml (e.g., at concentration of about 50 ng/ml).

According to some embodiments of the invention, culturing the epiblast cell and/or the late stage pluripotent stem cell is performed in a culture medium comprising a Wnt3a polypeptide and leukemia inhibitory factor (LIF).

According to some embodiments of the invention, the culture medium for culturing the epiblast cell and/or the late stage mammalian livestock pluripotent stem cells comprises the Wnt3a polypeptide at a concentration range between 5-50 ng/ml (e.g., at concentration of about 10 ng/ml), and LIF at a concentration range between 1000-3000 u/ml (e.g., at concentration of about 3000 u/ml). According to some embodiments of the invention, culturing the epiblast cell and/or the late stage pluripotent stem cell is performed in a culture medium comprising a Wnt3a polypeptide, basic fibroblast growth factor (bFGF) and leukemia inhibitory factor (LIF).

According to some embodiments of the invention, the culture medium for culturing the epiblast cell and/or the late stage mammalian livestock pluripotent stem cell comprises the Wnt3a polypeptide at a concentration range between 5-50 ng/ml (e.g., at concentration of about 10 ng/ml), bFGF at a concentration range between 20-100 ng/ml (e.g., at concentration of about 50 ng/ml) and LIF at a concentration range between 1000-3000 U/ml (e.g., at concentration of about 3000 U/ml).

According to some embodiments of the invention, the medium used for culturing the epiblast cell and/or the late stage pluripotent stem cell further comprises serum replacement.

As used herein the phrase “serum replacement” refers to a defined formulation, which substitutes the function of serum by providing pluripotent stem cells with components needed for growth and viability.

Various serum replacement formulations are known in the art and are commercially available.

For example, GIBCO™ Knockout™ Serum Replacement (Gibco-Invitrogen Corporation, Grand Island, N.Y. USA, Catalogue No. 10828028) is a defined serum-free formulation optimized to grow and maintain undifferentiated ES cells in culture. It should be noted that the formulation of GIBCO™ Knockout™ Serum Replacement includes Albumax (Bovine serum albumin enriched with lipids) which is from an animal source (International Patent Publication No. WO 98/30679 to Price, P. J. et al). However, a recent publication by Crook et al., 2007 (Crook J M., et al., 2007, Cell Stem Cell, 1: 490-494) describes six clinical-grade hESC lines generated using FDA-approved clinical grade foreskin fibroblasts in cGMP-manufactured Knockout™ Serum Replacement (Invitrogen Corporation, USA, Catalogue No. 04-0095).

According to some embodiments of the invention, the concentration of GIBCO™ Knockout™ Serum Replacement in the culture medium is in the range of from about 1% [volume/volume (v/v)] to about 50% (v/v), e.g., from about 5% (v/v) to about 40% (v/v), e.g., from about 5% (v/v) to about 30% (v/v), e.g., from about 10% (v/v) to about 30% (v/v), e.g., from about 10% (v/v) to about 25% (v/v), e.g., from about 10% (v/v) to about 20% (v/v), e.g., about 10% (v/v), e.g., about 15% (v/v), e.g., about 20% (v/v), e.g., about 30% (v/v).

Another commercially available serum replacement is the B27 supplement without vitamin A which is available from Gibco-Invitrogen, Corporation, Grand Island, N.Y. USA, Catalogue No. 12587-010. The B27 supplement is a serum-free formulation which includes d-biotin, fatty acid free fraction V bovine serum albumin (BSA), catalase, L-carnitine HCl, corticosterone, ethanolamine HCl, D-galactose (Anhyd.), glutathione (reduced), recombinant human insulin, linoleic acid, linolenic acid, progesterone, putrescine-2-HCl, sodium selenite, superoxide dismutase, T-3/albumin complex, DL alpha-tocopherol and DL alpha tocopherol acetate.

According to some embodiments of the invention the serum replacement is xeno-free.

For example, a xeno-free serum replacement can include a combination of insulin, transferrin and selenium.

Non-limiting examples of commercially available xeno-free serum replacement compositions include the premix of ITS (Insulin, Transferrin and Selenium) available from Invitrogen corporation (ITS, Invitrogen, Catalogue No. 51500-056); According to some embodiments of the invention, the xeno-free serum replacement formulations ITS (Invitrogen corporation) and SR3 (Sigma) are diluted in a 1 to 100 ratio in order to reach an xl working concentration.

According to some embodiments of the invention a suitable culture medium for culturing the mammalian livestock pluripotent stem cells of some embodiments of the invention in an undifferentiated state comprises a base medium such as DMEM\F12 or KO-DMEM (e.g., about 80% v/v), supplemented with serum (e.g., defined fetal bovine serum (FBS), e.g., about 20% v/v). According to some embodiments of the invention, the culture medium further comprises 1 mM L-glutamine, 0.1 mM β-mercaptoethanol, and 1% v/v non-essential amino acid stock. It is noted that this culture medium can support the undifferentiated growth of bovine PSC that are cultured on feeder cells such as MEFs, while being passaged every 5-10 days. However, when the bovine PSCs are cultured on MEFs or on feeder free culture systems at a high density (e.g., without passaging for at least 14 days), at least 25% of the bovine PSCs undergo a spontaneous differentiation into an adipogenic cell lineage. For example, if the bovine PSCs are cultured on MEFs or on feeder free culture systems at a high density (e.g., without passaging for at least 21 days), at least 50% of the bovine PSCs undergo a spontaneous differentiation into an adipogenic cell lineage.

According to some embodiments of the invention a suitable culture medium for culturing the mammalian livestock pluripotent stem cells of some embodiments of the invention in an undifferentiated state comprises a base medium such as DMEM\F12 or KO-DMEM (e.g., about 85% v/v), supplemented with ko-serum replacement (about 15% v/v), the IL6RIL6 chimera (at a concentration in the range of 50-150 pg/ml, e.g., a concentration of about 100 pg/ml), bFGF (at a concentration range of 40-60 ng/ml, e.g., at a concentration of about 50 ng/ml). According to some embodiments of the invention, the culture medium further comprises 1 mM L-glutamine, 0.1 mM β-mercaptoethanol, and 1% non-essential amino acid stock.

According to some embodiments of the invention a suitable culture medium for culturing the mammalian livestock pluripotent stem cells of some embodiments of the invention in an undifferentiated state comprises a base medium such as DMEM\F12 or KO-DMEM (e.g., about 85% v/v), supplemented with ko-serum replacement (about 15% v/v), the IL6RIL6 chimera (at a concentration in the range of 50-150 ng/ml, e.g., a concentration of about 100 ng/ml), bFGF (at a concentration range of 40-60 ng/ml, e.g., at a concentration of about 50 ng/ml). According to some embodiments of the invention, the culture medium further comprises 1 mM L-glutamine, 0.1 mM β-mercaptoethanol, and 1% v/v non-essential amino acid stock.

According to some embodiments of the invention a suitable culture medium for culturing the mammalian livestock pluripotent stem cells of some embodiments of the invention in an undifferentiated state comprises a base medium such as DMEM\F12 or KO-DMEM (e.g., at a concentration of about 85% v/v), supplemented with ko-serum replacement (e.g., at a concentration of about 15% v/v), WNT3A (at a concentration range of 5-50 ng/ml, e.g., at a concentration of about 10 ng/ml), bFGF (at a concentration range of 20-100 ng/ml, e.g., at a concentration of about 100 ng/ml or at a concentration of about 50 ng/ml), and leukemia inhibitory factor (LIF) (at a concentration range of 1000-3000 U/ml, e.g., at a concentration of about 3000 U/ml). According to some embodiments of the invention, the culture medium further comprises 1 mM L-glutamine, 0.1 mM β-mercaptoethanol, and 1% v/v non-essential amino acid stock.

While in culture, the epiblast cells or the late-stage pluripotent stem cells can be passaged in order to obtain a population of mammalian livestock pluripotent stem cells.

As used herein the term “passage” or “passaging” as used herein refers to splitting the cells in the culture vessel to 2 or more culture vessels, typically including addition of fresh culture medium. Passaging is typically done when the cells reach a certain density in culture.

According to some embodiments of the invention, passaging is performed by mechanical passaging.

As used herein the phrase “mechanical dissociation” refers to separating the pluripotent stem cell clumps to single cells by employing a physical force rather than an enzymatic activity.

For mechanical dissociation, a pellet of pluripotent stem cells (which may be achieved by centrifugation of the cells) or an isolated pluripotent stem cells clump can be dissociated by pipetting the cells up and down in a small amount of medium (e.g., 0.2-1 ml). For example, pipetting can be performed for several times (e.g., between 3-20 times) using a tip of a 200 μl or 1000 μl pipette.

Additionally or alternatively, mechanical dissociation of large pluripotent stem cells clumps can be performed using a device designed to break the clumps to a predetermined size. Such a device can be obtained from CellArtis Goteborg, Sweden. Additionally or alternatively, mechanical dissociation can be manually performed using a needle such as a 27 g needle (BD Microlance, Drogheda, Ireland) while viewing the clumps under an inverted microscope.

According to some embodiments of the invention, passaging is effected under conditions devoid of enzymatic dissociation.

According to some embodiments of the invention, the method further comprising mechanically passaging the population of mammalian livestock pluripotent stem cells for at least 2-6 passages, e.g., at least 2-5 passages, e.g., at least 2-4 passages, to thereby obtain a population enriched with the mammalian livestock pluripotent stem cells.

According to some embodiments of the invention, passaging is performed by enzymatic dissociation of cell clumps.

Enzymatic digestion of pluripotent stem cells clump(s) can be performed by subjecting the clump(s) or the colonies to an enzyme such as type IV Collagenase (Worthington biochemical corporation, Lakewood, N.J., USA) and/or Dispase (Invitrogen Corporation products, Grand Island N.Y., USA). The time of incubation with the enzyme depends on the size of cell clumps or the colonies present in the cell culture. Typically, when pluripotent stem cells cell clumps are dissociated every 5-7 days while in culture, incubation of 20-60 minutes with 1.5 mg/ml type IV Collagenase results in small cell clumps which can be further cultured in the undifferentiated state. Alternatively, pluripotent stem cells clumps can be subjected to incubation of about 25 minutes with 1.5 mg/ml type IV Collagenase followed by five minutes incubation with 1 mg/ml Dispase.

According to some embodiments of the invention, the method further comprises enzymatic passaging the population of mammalian livestock pluripotent stem cells for at least 2-6 passages, e.g., at least 2-5 passages, e.g., at least 2-4 passages, to thereby obtain a population enriched with the mammalian livestock pluripotent stem cells.

As used herein the phrase “population enriched with the mammalian livestock pluripotent stem cells” refers to a population of cells comprising at least 70% of mammalian livestock pluripotent stem cells.

According to some embodiments of the invention, the population enriched with mammalian livestock pluripotent stem cells comprises at least 71% undifferentiated and pluripotent mammalian livestock stem cells, e.g., at least 75%, at least 80%, at least 85%, at least 90%, at least 95%, at least 99%, or more undifferentiated and pluripotent mammalian livestock stem cells.

Once obtained, the population enriched with mammalian livestock pluripotent stem cells can be cultured while being serially passaged.

According to some embodiments of the invention, the population of pluripotent stem cells is expanded in an undifferentiated state for an extended time period while being serially passaged.

According to some embodiments of the invention, the extended time period is at least one two weeks, e.g., at least one month, e.g., at least 3, 4, 5, 6, 7 months or more while in culture.

According to some embodiments of the invention, once obtained, the mammalian livestock pluripotent stem cells can be frozen in liquid nitrogen using a freezing solution such as a solution consisting of 10% v/v dimethyl sulfoxide (DMSO) (e.g., can be obtained from Sigma, St Louis, Mo., USA), 10% v/v fetal bovine serum (FBS) (e.g., can be obtained from Hyclone, Utah, USA) and 80% v/v DMEM\F12 (e.g., can be obtained from Biological Industries, Israel).

According to some embodiments of the invention, the serial passaging of the population enriched with the mammalian livestock pluripotent stem cells is performed every 4-10 days, e.g., every 5-7 days.

According to some embodiments of the invention, passaging the population enriched with the mammalian livestock pluripotent stem cells is performed by enzymatic passaging (e.g., using type IV collagenase, Dispase, TryPLE trypsin).

According to some embodiments of the invention, passaging the population enriched with the mammalian livestock pluripotent stem cells is performed by mechanical passaging.

According to some embodiments of the invention, passaging the population enriched with the mammalian livestock pluripotent stem cells is performed by mechanical passaging devoid of enzymatic passaging.

Thus, the method of some embodiments of the invention results in a mammalian livestock pluripotent stem cell line comprising a population enriched with mammalian livestock pluripotent stem cells.

According to some embodiments of the invention, the cells of the population of mammalian livestock pluripotent stem cells are capable of differentiation into the endoderm, mesoderm and ectoderm embryonic germ layers.

Differentiation of the mammalian livestock pluripotent stem cells of some embodiments of the invention into the endoderm, mesoderm and ectoderm embryonic germ layers can be performed by direct differentiation in cell culture, by differentiation into embryoid bodies and/or by teratoma formations.

According to some embodiments of the invention, the cells of the population of mammalian livestock pluripotent stem cells are capable of differentiation into embryoid bodies.

As used herein the phrase “embryoid bodies” (EBs) refers to three dimensional multicellular aggregates of differentiated and undifferentiated cells derivatives of three embryonic germ layers.

Embryoid bodies are formed upon the removal of pluripotent stem cells from the conditions which maintain them in an undifferentiated state, such as feeder layers, feeder cells-free culture systems or a culture medium capable of maintaining the cells in an undifferentiated and pluripotent state. Removal of pluripotent stem cells from feeder cells or feeder-free matrices can be effected using type IV Collagenase treatment for a limited time. Following dissociation from the culturing surface, the cells are transferred to tissue culture plates containing a culture medium supplemented with serum and amino acids.

During the culturing period, EBs are further monitored for their differentiation state. Cell differentiation can be determined upon examination of cell or tissue-specific markers which are known to be indicative of differentiation. For example, EB-derived-differentiated cells may express the neurofilament 68 KD which is a characteristic marker of the ectoderm cell lineage.

The differentiation level of the EB cells can be monitored by following the loss of expression of OCT-4, and the increased expression level of other markers such as α-fetoprotein, NF-68 kDa, α-cardiac and albumin. Methods useful for monitoring the expression level of specific genes are well known in the art and include RT-PCR, semi-quantitative RT-PCR, Northern blot, RNA in situ hybridization, Western blot analysis and immunohistochemistry.

Teratomas

The pluripotent capacity of the pluripotent stem cells of some embodiments of the invention can also be confirmed by injecting the cells into SCID mice [Evans M J and Kaufman M (1983). Pluripotential cells grown directly from normal mouse embryos. Cancer Surv. 2: 185-208], which upon injection form teratomas. Teratomas are fixed using 4% v/v paraformaldehyde and histologically examined for the three germ layers (i.e., endoderm, mesoderm and ectoderm).

In addition to monitoring a differentiation state, stem cells are often also being monitored for karyotype, in order to verify cytological euploidity, wherein all chromosomes are present and not detectably altered during culturing. Cultured stem cells can be karyotyped using a standard Giemsa staining and compared to published karyotypes of the corresponding species.

It is well known in the art that pluripotent stem cells can be induced to differentiation into the adipogenic lineage by direct induction in the presence of effective amounts of adipogenic differentiation agents. For example, direct differentiation can be achieved by culturing the pluripotent stem cells in the presence of a bone morphogenic protein 4 (BMP4) essentially as described in Qi-Qun Tang, 2004 [Proc. Natl. Acad. Sci. U.S.A. 101(26): 9607-9611 “Commitment of C3H10T1/2 pluripotent stem cells to the adipocyte lineage” ]. Additionally or alternatively, pluripotent stem cells can be differentiated into adipogenic cells via embryoid bodies (EBs) differentiation. For example, 10-day old EBs can be plated on gelatin-coated plates with medium (e.g., DMEM/F12) comprising 20% v/v KSR (knockout serum replacement), and following additional 10 days the outgrowth are cultured in a medium containing DMEM/F12 and 10% v/v KSR supplemented with IBMX (1-Methyl-3-Isobutylxanthine; e.g., at a concentration of 0.5 mM), dexamethasone (e.g., 0.25 μM), T3 (e.g., 0.2 nM), insulin (e.g., 1 pg/ml), and Rosiglitazone (e.g., 1 μM), essentially as described in Tala Mohsen-Kanson et al., 2014 (Stem Cells, 32: 1459-1467), which is fully incorporated herein by reference.

As used herein the phrase “adipogenic differentiation agent” refers to a substance e.g., hormone and/or a chemical agent which when added to pluripotent stem cells in an in-vitro culture results in induction of differentiation of the cells towards the adipogenic cell lineage, ultimately resulting in the generation of adipocytes.

According to some embodiments of the invention, the adipogenic differentiation agent induces differentiation towards adipogenic lineage of pluripotent stem cells which are cultured in a two-dimensional culture system (e.g., on a matrix or on feeder cell layer(s)).

Non-limiting examples of known adipogenic differentiation agents include, but are not limited to, IBMX (1-Methyl-3-Isobutylxanthine, or 3-isobutyl-1-methylxanthine, which are interchangeably used herein), hydrocortisone, dexamethasone, BMP (bone morphogenic protein), T3 (triiodothyronine), indomethacin and fatty acids such as monounsaturated omega5 (e.g., Myristoleic acid), monounsaturated omega7 (e.g., Palmitoleic acid), monounsaturated omega 9 (e.g., Erucic acid, Elaidic acid, Oleic acid) or branched fatty acids (e.g., Phytanic acid and Pristanic acid) essentially as described in F. Mehta et al 2019 Sissel Beate Rønning (ed.), Myogenesis: Methods and Protocols, Methods in Molecular Biology, vol. 1889, Springer Science+Business Media, LLC, part of Springer Nature 2019.

Following are exemplary effective concentration ranges suitable for inducing adipogenic differentiation of pluripotent stem cell such as human ESCs or iPSCs. An adipogenic differentiation medium may comprise 0.01-1 mM of 3-isobutyl-1-methylxanthine, 0.1-10 μM of hydrocortisone, 0.01-1 mM of indomethacin, 0.4-0.6 mM IBMX, 0.2-0.3 μM dexamethasone, 0.15-0.3 nM T3, 1-2 pg/ml insulin, and 1-2 μM Rosiglitazone.

In contrast to the previously identified pluripotent stem cells, the mammalian livestock PSCs (e.g., bovine PSCs) of some embodiments of the invention can spontaneously differentiate into adipocytes, without the addition of any adipogenic differentiation agent (e.g., hormones or chemicals) which induce differentiation towards adipogenic-lineage.

Example 1 of the Examples section which follows, and FIGS. 6A-B show that the bovine pluripotent stem cells according to some embodiments of the invention are capable of spontaneous differentiation into adipocytes (fat cells) without the addition of any adipogenic differentiation agents (e.g., hormones or chemicals). The presence of adipocytes can be confirmed by visualization of oil drops positively stained with Oil Red staining.

The mammalian livestock PSCs of some embodiments of the invention can be cultured on feeder cells (e.g., MEFs feeder layers) in the presence of a culture medium such as “medium X” or the IL6RIL6 chimera medium (which is a serum-free medium), each of which is devoid of adipogenic differentiation agents, and spontaneously differentiate to adipocyte as background differentiation or when left without passaging for more than 10 days, e.g., more than 14 days.

Thus, the mammalian livestock PSCs of some embodiments of the invention are capable of differentiating into adipocytes in the absence of serum, i.e., in a serum-free culture medium.

According to some embodiments of the invention, the mammalian livestock pluripotent stem cells are capable of spontaneous differentiation into adipocytes in an absence of adipogenic differentiation agent(s).

According to some embodiments of the invention, the mammalian livestock pluripotent stem cells are capable of spontaneous differentiation into adipocytes in an absence of adipogenic differentiation agent(s) and in a serum-free culture medium.

The phrase “in the absence of” when used herein with respect to adipogenic differentiation agent(s) refers to being devoid of an effective amount of the adipogenic agent as described above.

It will be appreciated that a culture medium which is devoid of adipogenic differentiation agent(s) may comprise trace amounts of the adipogenic differentiation agent(s) which when added to a culture of human embryonic stem cells or human embryoid bodies without passaging for about 14-21 days cannot result in differentiation into adipocytes, since there is no effective amount.

According to some embodiments of the invention, the mammalian livestock pluripotent stem cells are capable of spontaneous differentiation into adipocytes when cultured without passaging for at least 10 days, e.g., more than 14 days in a medium devoid of dexamethasone.

According to some embodiments of the invention, the mammalian livestock pluripotent stem cells are capable of spontaneous differentiation into adipocytes when cultured without passaging for least 10 days, e.g., more than 14 days in a medium devoid of IBMX (1-Methyl-3-Isobutylxanthine).

According to some embodiments of the invention, the mammalian livestock pluripotent stem cells are capable of spontaneous differentiation into adipocytes when cultured without passaging for least 10 days, e.g., more than 14 days in a medium devoid of BMP.

According to some embodiments of the invention, the mammalian livestock pluripotent stem cells are capable of spontaneous differentiation into adipocytes when cultured without passaging for least 10 days, e.g., more than 14 days in a medium devoid of T3.

According to some embodiments of the invention, cells of the population of mammalian livestock pluripotent stem cells spontaneously differentiate into adipogenic cell lineage when cultured in a culture medium without passaging for about 10-14 days.

According to some embodiments of the invention, the culture medium used for the spontaneous differentiation into adipogenic lineage comprises serum.

According to some embodiments of the invention, the culture medium used for the spontaneous differentiation into adipogenic lineage comprises the IL6RIL6 chimera.

According to an aspect of some embodiments of the invention, there is provided an isolated mammalian livestock pluripotent stem cell generated by the method of some embodiments of the invention, wherein the isolated mammalian livestock pluripotent stem cell is capable of differentiating into the ectoderm, mesoderm and ectoderm embryonic germ layers, and is capable of spontaneous differentiation into adipogenic cells when cultured in a medium devoid of adipogenic differentiation agents.

According to some embodiments of the invention, the isolated mammalian livestock pluripotent stem cell is characterized by a positive expression of OCT4 (a marker of pluripotent stem cells).

According to an aspect of some embodiments of the invention, there is provided a method of generating an adipocyte, comprising culturing the isolated mammalian livestock pluripotent stem cell of some embodiments of the invention, or the population of mammalian livestock pluripotent stem cells obtained by the method of some embodiments of the invention in a culture medium devoid of adipogenic differentiation agents for at least 4 days and no more than 60 days without passaging, e.g., for at least 10 days and no more than 60 days without passaging, e.g., for at least 14 days and no more than 50 days without passaging, e.g., for at least 14 days and no more than 40 days without passaging, e.g., for at least 14 days and no more than 30 days without passaging, e.g., for at least 14 days and no more than 25 days without passaging, thereby generating the adipocyte.

As used herein the phrase “mammalian livestock” refers to a domesticated mammalian animal which is typically used as a source of food, such as meat and/or milk.

According to some embodiments of the invention, the mammalian livestock is a ruminant mammalian livestock.

According to some embodiments of the invention, the mammalian livestock is a non-ruminant mammalian livestock.

According to some embodiments of the invention, the ruminant mammalian livestock is selected from the group consisting of a Bovinae subfamily, sheep, goat, deer, and camel.

According to some embodiments of the invention, the ruminant mammalian livestock of the Bovinae subfamily is cattle or a yak.

According to some embodiments of the invention, the ruminant mammalian livestock of the Bovinae subfamily is cattle.

According to some embodiments of the invention, the cattle is buffalo, bison or cow (bovine).

According to some embodiments of the invention, the mammalian livestock is cow (bovine).

According to some embodiments of the invention, the cattle is cow (bovine).

According to some embodiments of the invention, the non-ruminant mammalian livestock is selected from the group pig, rabbit, and horse.

According to some embodiments of the invention, the mammalian livestock is horse.

According to an aspect of some embodiments of the invention there is provided a method of preparing a food product, comprising incorporating the adipocyte generated by the method of some embodiments of the invention with a food product, thereby preparing the food product.

According to some embodiments of the invention, the food product comprises a cultured meat or cultured cells which can be combined with other substances to result in cultured meat.

As used herein the term “cultured meat” refers to in-vitro cultured animal cells processed to impart an organoleptic sensation and texture of meat.

The cultured meat product may include a variety of cells, including but not limited to adipocytes, muscle cells, blood cells, cartilage cells, bone cells, connective tissue cells, fibroblasts and/or cardiomyocytes.

According to some embodiments of the invention, the in vitro cultured animal cells are mammalian livestock cells.

According to some embodiments of the invention, the in vitro cultured animal cells are bovine cells (though other cells can be included e.g., fish, porcine, and avian).

According to some embodiments of the invention, the in vitro cultured animal cells are horse cells (though other cells can be included e.g., fish, porcine, and avian). According to some embodiments of the invention, the in vitro cultured animal cells are adipocytes which were obtained by spontaneous differentiation of the mammalian livestock pluripotent stem cells of some embodiments of the invention.

According to some embodiments of the invention, the cultured meat is substantially free from any harmful microbial or parasitic contamination.

As mentioned, the cultured meat comprises the adipocytes that spontaneously differentiated from the mammalian livestock (e.g., bovine) pluripotent stem cells of some embodiments of the invention.

It should be noted that the fattier meat is generally tastier, but a greater fat content may pose a greater risk of adverse health consequences such as heart disease.

According to some embodiments of the invention, the cultured meat includes a ratio of muscle to fat cells that can be controlled to produce a meat product with optimal flavor and health effects. For example, such a ratio can be controlled by initial seeding of the desired cells in a culture or by controlling the differentiation of the mammalian livestock pluripotent stem cells into muscle, cartilage, blood or fat cells.

Differentiation may occur on supporting layers to support the structure and/or texture of the cultured meat.

According to some embodiments of the invention, aseptic techniques may be used to culture the cells resulting in meat products that are substantially free from harmful microbes such as bacteria, fungi, viruses, prions, protozoa, or any combination of the above. Harmful microbes may include pathogenic type microorganisms such as Salmonella, Campylobacter, E. coli 0156:H7, etc. Aseptic techniques may also be employed in packaging the meat products as they come off the biological production line. Such quality assurance may be monitored by standard assays for microorganisms or chemicals that are already known in the art. “Substantially free” means that the concentration of microbes or parasites is below a clinically significant level of contamination, i.e., below a level wherein ingestion would lead to disease or adverse health conditions.

According to some embodiments of the invention, other nutrients such as vitamins that are normally lacking in meat products from whole animals may be added to increase the nutritional value of the meat. This may be achieved either through straight addition of the nutrients to the growth medium or through genetic engineering techniques. For example, the gene or genes for enzymes responsible for the biosynthesis of a particular vitamin, such as Vitamin D, A, or the different Vitamin B complexes, may be transfected in the cultured muscle cells to produce the particular vitamin.

According to some embodiments of the invention, the meat product derived from the cultured cells in vitro may include different derivatives of meat products. These derivatives may be prepared, for example, by grounding or shredding the tissues grown in vitro and mixed with appropriate seasoning to make meatballs, fishballs, hamburger patties, etc. The derivatives may also be prepared from layers of tissues cut and spiced into, for example, beef jerky, ham, bologna, salami, etc. Thus, the meat products of the present invention may be used to generate any kind of food product originating from the meat of an animal.

According to an aspect of some embodiments of the invention there is provided a food product comprising the adipocyte generated by the method of some embodiments of the invention.

As mentioned, the mammalian livestock pluripotent stem cells of some embodiments of the invention can be induced to differentiation into various cell lineages and cell types. Following are non-limiting methods for differentiation of the mammalian livestock pluripotent stem cells of some embodiments of the invention.

Differentiation into red blood cells—Pluripotent stem cells can be induced to differentiation into hematopoietic cells, such as red blood cells using various protocols.

For example, differentiation into hematopoietic cells can be achieved via differentiation of the pluripotent stem cells into embryoid bodies (EBs).

Pluripotent stem cells can be induced to differentiation to hematopoietic cells by spontaneous differentiation into embryoid bodies (EBs), essentially as described in H. Lapillonne, et al., 2010 [haematologica, 95(10): 1651-1659; “Red blood cell generation from human induced pluripotent stem cells: perspectives for transfusion medicine” ], which is fully incorporated herewith in its entirety. Briefly, differentiation into EBs is performed in the presence of a culture medium such as Iscove's modified Dulbecco's medium—glutamax containing human plasma in the presence of stem cell factor (SCF, e.g., about 100 ng/mL), thrombopoietin (TPO, e.g., about 100 ng/mL), FLT3 ligand (e.g., about 100 ng/mL), recombinant human bone morphogenetic protein 4 (BMP4; e.g., about 10 ng/mL), recombinant human vascular endothelial growth factor (VEGF-A165; e.g., about 5 ng/mL), interleukin-3 (IL-3; e.g., about 5 ng/mL), interleukin-6 (IL-6; e.g., about 5 ng/mL) and erythropoietin (Epo; e.g., about 3 U/mL). Following about 20 days in culture the resulting embryoid bodies contain cells having early erythroid commitment. The cells of the EBs are then dissociated into single cells and further cultured in a culture medium containing plasma (e.g., about 10% v/v), insulin (e.g., about 10 μg/ml) and heparin (e.g., about 3 U/mL) and additional factors such as SCF (e.g., about 100 ng/mL), IL-3 (e.g., about 5 ng/mL) and Epo (e.g., about 3 U/mL). Following 8 days in culture the medium is replaced with a culture medium supplemented with SCF (e.g., about 100 ng/mL) and Epo (e.g., about 3 U/mL) for additional 3 days. From day 11 to 25 the cells can be cultured in a medium supplemented with Epo (3 U/mL). This protocol can result in definitive erythrocytes capable of maturation up to enucleated red blood cells containing fetal hemoglobin in a functional tetrameric form.

Alternatively, pluripotent stem cells can be directly differentiated into definite erythroblasts, essentially as described in Bin Mao et al. (2016, Stem Cell Reports, Vol. 7, pp 869-883), which is fully incorporated herein by reference in its entirety. Briefly, pluripotent stem cells which are cultured on a two-dimensional matrix or on feeder cells can be induced to differentiation into hematopoietic lineage by replacing the culture medium from an hPSCs maintenance medium to a hematopoiesis-inducing medium. For example, the hematopoiesis-inducing medium can be an Iscove's modified Dulbecco's medium (IMDM) supplemented with fetal bovine serum (FBS; e.g., about 10% v/v) (e.g., Hyclone), 1% v/v non-essential amino acids, ascorbic acid (e.g., about 50 mg/mL), and VEGF (Vascular endothelial growth factor; e.g., about 20 ng/mL), and culturing can be for a culturing period of about 10-12 days so as to form hematopoietic and erythroid progenitors. At days 10-12 the co-culture can be harvested and transferred to an ultra-low attachment plate with serum-free expansion medium supplemented with stem cell factor (SCF; e.g., about 100 ng/mL), interleukin-6 (IL-6; e.g., about 100 ng/mL), interleukin-3 (IL-3; e.g., about 5 ng/mL), fetal liver (e.g., about 10 ng/mL), thrombopoietin (TPO; e.g., about 10 ng/mL), erythropoietin (EPO; e.g., about 4 IU/mL), and VEGF (e.g., about 20 ng/mL) for 6 days, following which the cells are cultured for additional 7-8 days in a serum-free medium supplemented with stem cell factor, interleukin-3 (IL-3) and erythropoietin. Finally for maturation of the erythroblasts, the cells are cultured for about 1-2 weeks in serum-free RBC medium supplemented erythropoietin (EPO) essentially as described in Giarratana, M. C., 2005 (Nat. Biotechnol. 23, 69-74), which is fully incorporated herewith in its entirety. It is noted that the mature erythroblasts (derived from pluripotent stem cells) can be identified by the GPA+CD36^{low/+} which express higher levels of beta-globin along with a gradual loss of mesodermal and endothelial properties, and terminally suppressed CD36.

Additionally or alternatively it is noted that once CD34+ cells are obtained or isolated, enucleated red blood cells can be obtained under feeder-free culture conditions essentially as described in Kenichi Miharada et al., 2006 (“Efficient Enucleation of Erythroblasts Differentiated in Vitro From Hematopoietic Stem and Progenitor Cells”; Nat. Biotechnol. 24(10):1255-6), which is fully incorporated herein by reference in its entirety. Briefly, CD34+ cells are cultured in a culture medium containing stem cell factor (SCF), eruthropoietin (EPO), interleukin-3 (IL-3), vascular endothelial growth factor (VEGF) and insulin-like growth factor-II (IGF-II) for the first passage and then in a medium supplemented with only SCF and EPO for passages II and III, to thereby obtain about 77% of nucleated red blood cells.

Differentiation into cardiomyocytes—Pluripotent stem cells can be induced to differentiation into cardiomyocytes using various known methods such as those described in P. W. Burridge et al., (2014; Nat Methods. 11: 855-860; “Chemically defined generation of human cardiomycytes”); I. Batalov et al., (2015; Biomarker Insights 2015:10(S1); “Differentiation of Cardiomycytes from Human Pluripotent Stem Cells Using Monolayer Culture”); and P. W. Burridge et al. 2013 (Chapter 12 In: Methods in Molecular Biology 997; Uma Lakshmipathy and Mohan C. Vemuri Editors; Pluripotent Stem Cells, Methods and Protocols; “Highly Efficient Directed Differentiation of Human Induced Pluripotent Stem Cells into Cardiomyocytes”), each of which is fully incorporated herein by reference in its entirety. For example, for cardiomyocyte differentiation the pluripotent stem cells can be cultured in a conditioned medium, allowing formation of embryoid bodies (EBs), which can then be exposed to a serum containing medium (e.g., fetal bovine serum) for adhesion and formation of contracting cardiomyocytes.

Differentiation into Smooth muscle cells—Pluripotent stem cells can be induced to differentiation into smooth muscle cells using various known methods, such as using multipotent vasculogenic pericytes, which can successfully differentiate into smooth muscle cells, essentially as described in Dar A., et al., 2012 (Circulation. 125: 87-99; “Multipotent Vasculogenic Pericytes From Human Pluripotent Stem Cells Promote Recovery of Murine Ischemic Limb”), which is fully incorporated herein by reference in its entirety. Briefly, the pluripotent stem cells undergo spontaneous differentiation into EBs and cells of the EBs which are CD105+/CD90+/CD73+/CD31-multipotent clonogenic mesodermal precursors can be isolated by MACS MicroBeads and give rise to pericytes, which can further proliferate and further differentiated into smooth muscle cells.

Additionally or alternatively, pluripotent stem cells can be cultured in a chemically defined culture medium comprising inhibitors of phosphoinositide 3-kinase (PI3K) and glycogen synthase kinase 3b(GSK3b) and the addition of bone morphogenic protein 4 (BMP4) and fibroblast growth factor 2 (FGF2), to successfully convert up to about 60% of the cells into the myogenic program by day 36 as indicated by MYOG+ cell populations, essentially as described in ELLIOT W. SWARTZ, et al., 2016 (“A Novel Protocol for Directed Differentiation of C9orf72-AssociatedHumanInduced Pluripotent Stem Cells Into Contractile Skeletal Myotubes”; STEM CELLS TRANSLATIONAL MEDICINE 2016; 5:1461-1472), which is fully incorporated herein by reference in its entirety.

Additional suitable methods of inducing differentiation of pluripotent stem cells into muscle cells are described in Jërome Chal et al., 2016 (“Generation of human muscle fibers and satellite-like cells from human pluripotent stem cells in vitro”; Nature protocols; VOL. 11: 1833-1850); Nunnapas Jiwlawat et al., 2018 (“Current Progress and Challenges for Skeletal Muscle Differentiation from Human Pluripotent Stem Cells Using Transgene-Free Approaches”; Stem Cells International, Volume 2018, pp: 1-18), each of which is fully incorporated herein by reference in its entirety.

Differentiation into cartilage cells—Pluripotent stem cells can be induced to differentiation into cartilage cells via formation of embryoid bodies, e.g., essentially as described in Sergey P. Medvedev et al., 2011 (“Human Induced Pluripotent Stem Cells Derived from Fetal Neural Stem Cells Successfully Undergo Directed Differentiation into Cartilage”; STEM CELLS AND DEVELOPMENT, Volume 20, Number 6: 1099-1112), which is fully incorporated herein by reference in its entirety. Briefly, pluripotent stem cells are allowed to spontaneously differentiate into embryoid bodies for 8-15 days. For directed chondrogenic differentiation, the embryoid bodies can be further cultivated for 21 days in a chondrogenic medium comprising DMEM, supplemented with bovine serum (e.g., about 5% v/v), dexamethasone (e.g., about 10 nM), ascorbic acid (e.g., about 50 μg/mL), L-proline (e.g., about 40 μg/mL), transforming growth factor b3 (TGFβ3; e.g., about 10 ng/mL) and bone morphogenetic protein-2 (BMP2; e.g., about 10 ng/mL). For a further cartilage self-assembly, the EBs can be disaggregated (e.g., using trypsin), and further transferred to coated 96-well plates (e.g., coated with agarose), at a density of 10⁵ cells per well and further cultured in the same medium.

Additionally or alternatively, pluripotent stem cells can be directly differentiated into chondrocytes by plating the cells on a matrix in the presence of a chondrogenic-inducing culture medium, using various protocols, for example, as reviewed in Michal Lach et al., 2014. Journal of Tissue Engineering Volume 5: 1-9, which is fully incorporated herein by reference in its entirety. For example, pluripotent stem cells can be cultured on a matrix in a medium supplemented with various growth factors such as WNT-3a, activin, follistatin, BMP4, fibroblast growth factor 2 (FGF2), growth and differentiation factor 5 (GDF5) and neurotrophin 4 (NT4), essentially as described in Oldershaw R A, et al. 2010 (“Directed differentiation of human embryonic stem cells toward chondrocytes”; Nat Biotechnol 28(11): 1187-1194), which is fully incorporated herein by reference in its entirety.

Additionally or alternatively, pluripotent stem cells can be cultured in a medium comprising only six growth factors WNT-3a, activin, follistatin, BMP4, fibroblast growth factor 2 (FGF2), and growth and differentiation factor 5 (GDF5) essentially as described in Yang S-L, et al. 2012 (“Compound screening platform using human induced pluripotent stem cells to identify small molecules that promote chondrogenesis”. Protein Cell, 3(12): 934-942), which is fully incorporated herein by reference in its entirety. These protocols can result in differentiation into chondrocyte-like cells with high COL2A1 (Collagen type II, alpha 1) and SRY (sex determining region Y)-box 9 (SOX9) expression and decreased pluripotent marker expression compared to control cell lines.

Neural Precursor Cells

To differentiate the EBs of some embodiments of the invention into neural precursors, four-day-old EBs are cultured for 5-12 days in tissue culture dishes including DMEM/F-12 medium with 5 mg/ml insulin, 50 mg/ml transferrin, 30 nM selenium chloride, and 5 mg/ml fibronectin (ITSFn medium, Okabe, S. et al., 1996, Mech. Dev. 59: 89-102). The resultant neural precursors can be further transplanted to generate neural cells in vivo (Brustle, O. et al., 1997. In vitro-generated neural precursors participate in mammalian brain development. Proc. Natl. Acad. Sci. USA. 94: 14809-14814). It will be appreciated that prior to their transplantation, the neural precursors are trypsinized and triturated to single-cell suspensions in the presence of 0.1% DNase.

Oligodendrocytes and myelinate cells EBs of some embodiments of the invention can differentiate to oligodendrocytes and myelinate cells by culturing the cells in modified SATO medium, i.e., DMEM with bovine serum albumin (BSA), pyruvate, progesterone, putrescine, thyroxine, triiodothryonine, insulin, transferrin, sodium selenite, amino acids, neurotrophin 3, ciliary neurotrophic factor and Hepes (Bottenstein, J. E. & Sato, G. H., 1979, Proc. Natl. Acad. Sci. USA 76, 514-517; Raff, M. C., Miller, R. H., & Noble, M., 1983, Nature 303: 390-396]. Briefly, EBs are dissociated using 0.25% v/v Trypsin/EDTA (5 min at 37° C.) and triturated to single cell suspensions. Suspended cells are plated in flasks containing SATO medium supplemented with 5% v/v equine serum and 5% v/v fetal calf serum (FCS). Following 4 days in culture, the flasks are gently shaken to suspend loosely adhering cells (primarily oligodendrocytes), while astrocytes are remained adhering to the flasks and further producing conditioned medium. Primary oligodendrocytes are transferred to new flasks containing SATO medium for additional two days. Following a total of 6 days in culture, oligospheres are either partially dissociated and resuspended in SATO medium for cell transplantation, or completely dissociated and a plated in an oligosphere-conditioned medium which is derived from the previous shaking step [Liu, S. et al., (2000). Embryonic stem cells differentiate into oligodendrocytes and myelinate in culture and after spinal cord transplantation. Proc. Natl. Acad. Sci. USA. 97: 6126-6131].

Mast Cells

For mast cell differentiation, two-week-old EBs of some embodiments of the invention are transferred to tissue culture dishes including DMEM medium supplemented with 10% v/v FCS, 2 mM L-glutamine, 100 units/ml penicillin, 100 mg/ml streptomycin, 20% (v/v) WEHI-3 cell-conditioned medium and 50 ng/ml recombinant rat stem cell factor (rrSCF, Tsai, M. et al., 2000. In vivo immunological function of mast cells derived from embryonic stem cells: An approach for the rapid analysis of even embryonic lethal mutations in adult mice in vivo. Proc Natl Acad Sci USA. 97: 9186-9190). Cultures are expanded weekly by transferring the cells to new flasks and replacing half of the culture medium.

Hemato-Lymphoid Cells

To generate hemato-lymphoid cells from the EBs of some embodiments of the invention, 2-3 days-old EBs are transferred to gas-permeable culture dishes in the presence of 7.5% CO₂ and 5% 02 using an incubator with adjustable oxygen content. Following 15 days of differentiation, cells are harvested and dissociated by gentle digestion with Collagenase (0.1 unit/mg) and Dispase (0.8 unit/mg), both are available from F. Hoffman-La Roche Ltd, Basel, Switzerland. CD45-positive cells are isolated using anti-CD45 monoclonal antibody (mAb) M1/9.3.4.HL.2 and paramagnetic microbeads (Miltenyi) conjugated to goat anti-rat immunoglobulin as described in Potocnik, A. J. et al., (Immunology Hemato-lymphoid in vivo reconstitution potential of subpopulations derived from in vitro differentiated embryonic stem cells. Proc. Natl. Acad. Sci. USA. 1997, 94: 10295-10300). The isolated CD45-positive cells can be further enriched using a single passage over a MACS column (Miltenyi).

It will be appreciated that since EBs are complex structures, differentiation of EBs into specific differentiated cells, tissue or organ may require isolation of lineage specific cells from the EBs.

Such isolation may be effected by sorting of cells of the EBs via fluorescence activated cell sorter (FACS) or mechanical separation of cells, tissues and/or tissue-like structures contained within the EBs.

Methods of isolating EB-derived-differentiated cells via FACS analysis are known in the art. According to one method, EBs are disaggregated using a solution of Trypsin and EDTA (0.025% v/v and 0.01% v/v, respectively), washed with 5% v/v fetal bovine serum (FBS) in phosphate buffered saline (PBS) and incubated for 30 min on ice with fluorescently-labeled antibodies directed against cell surface antigens characteristics to a specific cell lineage. For example, endothelial cells are isolated by attaching an antibody directed against the platelet endothelial cell adhesion molecule-1 (PECAMI) such as the fluorescently-labeled PECAMI antibodies (30884X) available from PharMingen (PharMingen, Becton Dickinson Bio Sciences, San Jose, Calif., USA) as described in Levenberg, S. et al., (Endothelial cells derived from human embryonic stem cells. Proc. Natl. Acad. Sci. USA. 2002. 99: 4391-4396). Hematopoietic cells are isolated using fluorescently-labeled antibodies such as CD34-FITC, CD45-PE, CD31-PE, CD38-PE, CD90-FITC, CD117-PE, CD15-FITC, class I-FITC, all of which IgG1 are available from PharMingen, CD133/1-PE (IgG1) (available from Miltenyi Biotec, Auburn, Calif.), and glycophorin A-PE (IgG1), available from Immunotech (Miami, Fla.). Live cells (i.e., without fixation) are analyzed on a FACScan (Becton Dickinson Bio Sciences) by using propidium iodide to exclude dead cells with either the PC-LYSIS or the CELLQUEST software. It will be appreciated that isolated cells can be further enriched using magnetically-labeled second antibodies and magnetic separation columns (MACS, Miltenyi) as described by Kaufman, D. S. et al., (Hematopoietic colony-forming cells derived from human embryonic stem cells. Proc. Natl. Acad. Sci. USA. 2001, 98: 10716-10721).

An example for mechanical isolation of beating cardiomyocytes from EBs is disclosed in U.S. Pat. Appl. No. 20030022367 to Xu et al. Briefly, four-day-old EBs of some embodiments of the invention are transferred to gelatin-coated plates or chamber slides and are allowed to attach and differentiate. Spontaneously contracting cells, which are observed from day 8 of differentiation, are mechanically separated and collected into a 15-mL tube containing low-calcium medium or PBS. Cells are dissociated using Collagenase B digestion for 60-120 minutes at 37° C., depending on the Collagenase activity. Dissociated cells are then resuspended in a differentiation KB medium (85 mM KCI, 30 mM K₂HPO₄, 5 mM MgSO₄, 1 mM EGTA, 5 mM creatine, 20 mM glucose, 2 mM Na₂ATP, 5 mM pyruvate, and 20 mM taurine, buffered to pH 7.2, Maltsev et al., Circ. Res. 75:233, 1994) and incubated at 37° C. for 15-30 min. Following dissociation cells are seeded into chamber slides and cultured in the differentiation medium to generate single cardiomyocytes capable of beating.

It will be appreciated that the culturing conditions suitable for the differentiation and expansion of the isolated lineage specific cells include various tissue culture medium, growth factors, antibiotic, amino acids and the like and it is within the capability of one skilled in the art to determine which conditions should be applied in order to expand and differentiate particular cell types and/or cell lineages [reviewed in Fijnvandraat A C, et al., Cardiovasc Res. 2003; 58: 303-12; Sachinidis A, et al., Cardiovasc Res. 2003; 58: 278-91; Stavridis M P and Smith A G, 2003; Biochem Soc Trans. 31(Pt 1): 45-9].

Cell lines of some embodiments of the invention can be produced by immortalizing the EB-derived cells by methods known in the art, including, for example, expressing a telomerase gene in the cells (Wei, W. et al., 2003. Mol Cell Biol. 23: 2859-2870) or co-culturing the cells with NIH 3T3 hph-HOX11 retroviral producer cells (Hawley, R. G. et al., 1994. Oncogene 9: 1-12).

Following are non-limiting examples of culturing conditions which are suitable for differentiating and/or expanding lineage specific cells from pluripotent stem cells (e.g., ESCs and iPS cells).

Mesenchymal stromal cells which are CD73-positive and SSEA-4-negative can be generated from pluripotent stem cells by mechanically increasing the fraction of fibroblast-like differentiated cells formed in cultures of pluripotent stem cells, essentially as described in Trivedi P and Hematti P. Exp Hematol. 2008, 36(3):350-9. Briefly, to induce differentiation of pluripotent stem cells the intervals between medium changes are increased to 3-5 days, and the cells at the periphery of the ESC colonies become spindle-shaped fibroblast-looking cells. After 9-10 days under these conditions when about 40-50% of the cells in the culture acquire the fibroblast-looking appearance, the undifferentiated portions of pluripotent stem cells colonies are physically removed and the remaining differentiated cells are passaged to new culture plates under the same conditions.

To induce differentiation of pluripotent stem cells into dopaminergic (DA) neurons, the cells can be co-cultured with the mouse stromal cell lines PA6 or MS5, or can be cultured with a combination of stromal cell-derived factor 1 (SDF-1/CXCL12), pleiotrophin (PTN), insulin-like growth factor 2 (IGF2) and ephrin B1 (EFNB1) essentially as described in Vazin T, et al., PLoS One. 2009 Aug. 12; 4(8):e6606; and in Elkabetz Y., et al., Genes Dev. 2008 January 15; 22: 152-165.

To generate mesencephalic dopamine (mesDA) neurons, pluripotent stem cells can be genetically modified to express the transcription factor Lmxla (e.g., using a lentiviral vector with the PGK promoter and Lmxla) essentially as described in Friling S., et al., Proc Natl Acad Sci USA. 2009, 106: 7613-7618.

To generate lung epithelium (type II pneumocytes) from pluripotent stem cells, the pluripotent stem cells can be cultured in the presence of a commercially available cell culture medium (Small Airway Growth Medium; Cambrex, College Park, Md.), or alternatively, in the presence of a conditioned medium collected from a pneumocyte cell line (e.g., the A549 human lung adenocarcinoma cell line) as described in Rippon H J., et al., Proc Am Thorac Soc. 2008; 5: 717-722.

To induce differentiation of pluripotent stem cells into neural cells, the pluripotent stem cells can be cultured for about 5 days in the presence of a serum replacement medium supplemented with TGF-b inhibitor (SB431542, Tocris; e.g., 10 nM) and Noggin (R&D; e.g., 500 ng/ml), following which the cells are cultured with increasing amounts (e.g., 25%, 50%, 75%, changed every two days) of N2 medium (Li X J., et al., Nat Biotechnol. 2005, 23:215-21) in the presence of 500 ng/mL Noggin, essentially as described in Chambers S M., et al., Nat Biotechnol. 2009, 27: 275-280.

In addition to the lineage-specific primary cultures, EBs of the invention can be used to generate lineage-specific cell lines which are capable of unlimited expansion in culture.

Cell lines of some embodiments of the invention can be produced by immortalizing the EB-derived cells by methods known in the art, including, for example, expressing a telomerase gene in the cells (Wei, W. et al., 2003. Mol Cell Biol. 23: 2859-2870) or co-culturing the cells with NIH 3T3 hph-HOX11 retroviral producer cells (Hawley, R. G. et al., 1994. Oncogene 9: 1-12).

As used herein the term “IL6RIL6 chimera” refers to a chimeric polypeptide which comprises the soluble portion of interleukin-6 receptor (IL-6-R, e.g., the human IL-6-R as set forth by GenBank Accession No. AAH89410; SEQ ID NO: 1) (e.g., a portion of the soluble IL6 receptor as set forth by amino acids 112-355 (SEQ ID NO: 2) of GenBank Accession No. AAH89410) and the interleukin-6 (IL6) (e.g., human IL-6 as set forth by GenBank Accession No. CAG29292; SEQ ID NO: 3) or a biologically active fraction thereof (e.g., a receptor binding domain). Preferably, the IL6RIL6 chimera used by the method according to this aspect of the present invention is capable of supporting the undifferentiated growth of human embryonic stem cells, while maintaining their pluripotent capacity. It will be appreciated that when constructing the IL6RIL6 chimera the two functional portions (i.e., the IL6 and its receptor) can be directly fused (e.g., attached or translationally fused, i.e., encoded by a single open reading frame) to each other or conjugated (attached or translationally fused) via a suitable linker (e.g., a polypeptide linker). Preferably, the IL6RIL6 chimeric polypeptide exhibits a similar amount and pattern of glycosylation as the naturally occurring IL6 and IL6 receptor. For example, a suitable IL6RIL6 chimera is as set forth in SEQ ID NO:4 and in FIG. 11 of WO 99/02552 to Revel M., et al., which is fully incorporated herein by reference.

As used herein the term “WNT3A” refers to a member of the WNT gene family. The WNT gene family consists of structurally related genes which encode secreted signaling proteins. These proteins have been implicated in oncogenesis and in several developmental processes, including regulation of cell fate and patterning during embryogenesis.

The WNT3A mRNA (GenBank Accession NO. NM_033131.3; SEQ ID NO:5) encodes the WNT3A polypeptide (GenBank Accession No. NP_149122.1; SEQ ID NO: 6). The WNT3A polypeptide can be obtained from various manufacturers such as R&D SYSTEMS (e.g., Catalogue No. 5036-WN-010).

As used herein the term “basic fibroblast growth factor (bFGF)” refers to a polypeptide of the fibroblast growth factor (FGF) family, which binds heparin and possesses broad mitogenic and angiogenic activities. The mRNA for the BFGF gene contains multiple polyadenylation sites, and is alternatively translated from non-AUG (CUG) and AUG initiation codons, resulting in five different isoforms with distinct properties. The CUG-initiated isoforms are localized in the nucleus and are responsible for the intracrine effect, whereas, the AUG-initiated form is mostly cytosolic and is responsible for the paracrine and autocrine effects of this FGF.

The bFGF polypeptide is provided in GenBank Accession No. NP_001997 (SEQ ID NO:7), which can be obtained from various manufacturers such as Peprotech, R&D systems (e.g., Catalog Number: 233-FB), and Millipore.

Bovine bFGF polypeptide is provided in GenBank Accession No. NP_776481.2 (SEQ ID NO: 11), encoded by SEQ ID NO: 12 (GenBank Accession No. NM_174056.4). Bovine bFGF can be obtained from R & D systems, e.g., bovine FGF basic/FGF2/bFGF (From Bovine Brain; Cat. No. 133-FB-025) or Recombinant Bovine FGF basic/FGF2/bFGF (E. Coli derived; Cat. No. 2099-FB-025). As used herein the term “leukemia inhibitory factor (LIF)” refers to the pleiotropic cytokine which is involved in the induction of hematopoietic differentiation, induction of neuronal cell differentiation, regulator of mesenchymal to epithelial conversion during kidney development, and may also have a role in immune tolerance at the maternal-fetal interface.

The LIF used in the culture medium of some embodiments of the invention can be a purified, synthetic or recombinantly expressed LIF protein [e.g., human LIF polypeptide GenBank Accession No. NP_002300.1 (SEQ ID NO:8); human LIF polynucleotide GenBank Accession No. NM_002309.4 (SEQ ID NO:9); bovine LIF polypeptide GenBank Accession No. NP_776356.1 (SEQ ID NO: 10), encoded by GenBank Accession No. NM_173931.1 (SEQ ID NO: 11)]. It should be noted that for the preparation of a xeno-free culture medium LIF is preferably recombinantly expressed. Recombinant human LIF can be obtained from various sources such as Chemicon, USA (Catalogue No. LIF10100) and AbD Serotec (MorphoSys US Inc, Raleigh, N.C. 27604, USA). Murine LIF ESGRO® (LIF) can be obtained from Millipore, USA (Catalogue No. ESG1107).

According to some embodiments of the invention, the concentration of LIF in the culture medium is from about 1000 units/ml to about 10,000 units/ml, e.g., from about 2000 units/ml to about 10,000 units/ml, e.g., from about 2000 units/ml to about 8,000 units/ml, e.g., from about 2000 units/ml to about 6,000 units/ml, e.g., from about 2000 units/ml to about 5,000 units/ml, e.g., from about 2000 units/ml to about 4,000 units/ml, e.g., from about 2,500 units/ml to about 3,500 units/ml, e.g., from about 2,800 units/ml to about 3,200 units/ml, e.g., from about 2,900 units/ml to about 3,100 units/ml, e.g., about 3000 units/ml.

According to some embodiments of the invention, the concentration of LIF in the culture medium is at least about 1000 units/ml, e.g., at least about 2000 units/ml, e.g., at least about 2100 units/ml, e.g., at least about 2200 units/ml, e.g., at least about 2300 units/ml, e.g., at least about 2400 units/ml, e.g., at least about 2500 units/ml, e.g., at least about 2600 units/ml, e.g., at least about 2700 units/ml, e.g., at least about 2800 units/ml, e.g., at least about 2900 units/ml, e.g., at least about 2950 units/ml, e.g., about 3000 units/ml.

As mentioned, any of the proteinaceous factors used in the culture medium of the present invention (e.g., the bFGF, IL6RIL6 chimera, WNT3a, LIF) can be recombinantly expressed or biochemically synthesized. In addition, naturally occurring proteinaceous factors such as bFGF, WNT3a, LIF can be purified from biological samples (e.g., from human serum, cell cultures) using methods well known in the art. It should be noted that for the preparation of xeno-free culture medium the proteinaceous factor is preferably recombinantly expressed.

Biochemical synthesis of the proteinaceous factors of the present invention can be performed using standard solid phase techniques. These methods include exclusive solid phase synthesis, partial solid phase synthesis methods, fragment condensation and classical solution synthesis.

Recombinant expression of the proteinaceous factors of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680, Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463. Specifically, the IL6RIL6 chimera can be generated as described in PCT publication WO 99/02552 to Revel M., et al. and Chebath J, et al., 1997, which are fully incorporated herein by reference.

As mentioned, the method of some embodiments of the invention employs culturing the mammalian livestock (e.g., bovine) embryos or the stem cells on feeder cell layers or on feeder cell-free culture systems.

Following are exemplary, non-limiting descriptions of feeder cell layers.

Mouse feeder layers—The most common method for culturing pluripotent stem cells is based on mouse embryonic fibroblasts (MEF) as a feeder cell layer supplemented with tissue culture medium containing serum or leukemia inhibitor factor (LIF) which supports the proliferation and the pluripotency of the pluripotent stem cells [Thomson J A, Itskovitz-Eldor J, Shapiro S S, Waknitz M A, Swiergiel J J, Marshall V S, Jones J M. (1998). Embryonic stem cell lines derived from human blastocysts. Science 282: 1145-7; Reubinoff B E, Pera M F, Fong C, Trounson A, Bongso A. (2000). Embryonic stem cell lines from human blastocysts: somatic differentiation in vitro. Nat. Biotechnol. 18: 399-404]. MEF cells are derived from day 12-13 mouse embryos in medium supplemented with fetal bovine serum. Under these conditions mouse ES cells can be maintained in culture as pluripotent stem cells, preserving their phenotypical and functional characteristics. It should be noted that the use of feeder cells substantially increases the cost of production. Additionally, the feeder cells are metabolically inactivated to keep them from outgrowing the stem cells, hence it is necessary to have fresh feeder cells for each splitting of pluripotent stem cell culture.

Pluripotent stem cells can also be cultured on MEF under serum-free conditions using serum replacement supplemented with basic fibroblast growth factor (bFGF) [Amit M, Carpenter M K, Inokuma M S, Chiu C P, Harris C P, Waknitz M A, Itskovitz-Eldor J, Thomson J A. (2000). Clonally derived human embryonic stem cell lines maintain pluripotency and proliferative potential for prolonged periods of culture. Dev. Biol. 227: 271-8]. Under these conditions the cloning efficiency of ES cells is 4 times higher than under fetal bovine serum. In addition, following 6 months of culturing under serum replacement the ES cells still maintain their pluripotency as indicated by their ability to form teratomas which contain all three embryonic germ layers. Although this system uses a better-defined culture conditions, the presence of mouse cells in the culture may expose the pluripotent stem cell culture to mouse pathogens which restricts their use in cell-based therapy.

Human embryonic fibroblasts or adult fallopian epithelial cells as feeder cell layers—Embryonic stem cells can be grown and maintained using human embryonic fibroblasts or adult fallopian epithelial cells. When grown on these human feeder cells the embryonic stem cells exhibit normal karyotypes, present alkaline phosphatase activity, express Oct-4 and other embryonic cell surface markers including SSEA-3, SSEA-4, TRA-1-60, and GCTM-2, form teratomas in vivo, and retain all key morphological characteristics [Richards M, Fong C Y, Chan W K, Wong P C, Bongso A. (2002). Human feeders support prolonged undifferentiated growth of human inner cell masses and embryonic stem cells. Nat. Biotechnol. 20: 933-6].

Foreskin feeder layers—Embryonic stem cells can be cultured on human foreskin feeder layer as disclosed in U.S. patent application Ser. No. 10/368,045. Foreskin derived feeder cell layers consist of a complete animal-free environment suitable for culturing embryonic stem cells. In addition, foreskin cells can be maintained in culture for as long as 42 passages since their derivation, providing the embryonic stem cells with a relatively constant environment. Under these conditions the embryonic stem cells were found to be functionally indistinct from cells grown with alternate protocols (e.g., MEF). Following differentiation, embryonic stem cells expressed genes associated with all three embryonal germ layers, in vitro, and formed teratomas in vivo, consisting of tissue arising from all three germ layers. In addition, unlike human fallopian epithelial cells or human embryonic fibroblasts, human embryonic stem cells cultured on foreskin feeder layers were maintained in culture in a pluripotent and undifferentiated state for at least 87 passages. However, although foreskin cells can be maintained in culture for long periods (i.e., 42 passages), the foreskin culture system is not well-defined due to differences between separate batches. In addition, human feeder layer-based culture systems would still require the simultaneous growth of both feeder layers and hES cells. Therefore, feeder-free culturing systems have been developed.

Following are exemplary, non-limiting descriptions of feeder-free culture systems.

Stem cells can be grown on a solid surface such as an extracellular matrix (e.g., Matrigel™ or laminin) in the presence of a culture medium. Unlike feeder-based cultures which require the simultaneous growth of feeder cells and stem cells and which may result in mixed cell populations, stem cells grown on feeder-free systems are easily separated from the surface. The culture medium used for growing the stem cells contains factors that effectively inhibit differentiation and promote their growth such as MEF-conditioned medium and bFGF. However, commonly used feeder-free culturing systems utilize an animal-based matrix (e.g., Matrigel™) supplemented with mouse or bovine serum, or with MEF conditioned medium [Xu C, et al. (2001). Feeder-free growth of undifferentiated human embryonic stem cells. Nat Biotechnol. 19: 971-4]which present the risk of animal pathogen cross-transfer to the human ES cells, thus compromising future clinical applications.

According to some embodiments of the invention, the feeder-free matrix is selected from the group consisting of a Matrigel™ matrix, a fibronectin matrix, a laminin matrix, and a vitronectin matrix.

The pluripotent stem cells of some embodiments of the invention, or the cells differentiated therefrom (e.g., adipocytes, muscle cells, blood cells, cartilage cells, bone cells, connective tissue cells, fibroblasts and/or cardiomyocytes) can be identified using various expression markers characterizing these cells. The expression markers can be identified on the RNA or protein level.

Methods of detecting the expression level of RNA include, but are not limited to Northern Blot analysis, RT-PCR analysis, RNA in situ hybridization stain, In situ RT-PCR stain, DNA microarrays/DNA chips, and Oligonucleotide microarray.

Methods of detecting expression and/or activity of proteins include, but are not limited to Enzyme linked immunosorbent assay (ELISA), Western blot, Radio-immunoassay (RIA),

Fluorescence activated cell sorting adipocytes, muscle cells, blood cells, cartilage cells, bone cells, connective tissue cells, fibroblasts and/or cardiomyocytes (FACS), Immunohistochemical analysis, and In situ activity assay.

As used herein the term “about” refers to ±10%.

According to some embodiments of the invention, the tem “about” refers to ±9%, 8%, ±7%, 6%, 5%, 4%, 3%, 2%, 1%, 0.5%, 0.1%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 13 is expressed in a DNA sequence format (e.g., reciting T for thymine), but it can refer to either a DNA sequence that corresponds to an bovine bFGF nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Methods

One of the differences between bovine and horse delayed blastocysts is that in bovine the embryos are obtained from cows at day 7 post insemination (compacted embryo or early blastocyst) while with mares the embryo is obtained at day 8 post insemination (blastocyst or expended blastocyst, usually only one embryo).

Blastocyst Cultivation from Bovine:

Seven days-old embryos were obtained from cows (Holstein Friesian cattle) undergoing in uterus fertilization followed by uterus washing. Embryos were washed and maintained using Holding and transfer medium (BioLife, Item C15C, USA) till there were transferred to culture conditions (up to one hour).

Blastocysts can also be obtained from the following source:
Commercially available (Sion, Ha'fetz Ha'im, Israel)
IVF, oocyte insemination in vitro
Nuclear Transfer (NT) of Bovine cell.
Parthenogenesis

Derivation of Bovine PSC Lines:

After zona pellucida digestion by Tyrode's acidic solution (Sigma Aldrich, St Louis, Mo., USA) the exposed blastocysts were plated. Two different plating methods were employed:

(i) on feeder layer, such as mitotically inactivated mouse embryonic fibroblasts (MEFs) or mitotically inactivated foreskin fibroblasts,

(ii) on suitable matrix (Matrigel™ matrix, Fibronectin, Laminin, Vitronectin, commercial cell matrices).

The embryos were attached to the surface using any one of the following techniques:

(i) using a 27 g needle;

(ii) using a pulled Pasteur Pipette;

(iii) by covering the embryo with a drop of a suitable matrix;

(iv) or by leaving the embryo on a plate until the embryo is spontaneously attached to the surface.

Attached bovine blastocysts are cultured on MEFs as whole embryos for 7-21 days post fertilization until a large cyst is developed (e.g., 14 days post insemination as shown in FIG. 1C). If needed due to the MEF or matrix quality, the embryos are transferred in whole to new MEF-covered plates using 27 gouge syringe needles, leaving a few of the surrounding fibroblasts behind. After the embryo develops a cyst, a disc-like structure is isolated from it and plated separately on a fresh MEF or matrix-covered plate. Cells with stem cell morphology (small cells with large nucleus) are passaged mechanically. After a few passages (4-6 passages), when a population enriched with bovine pluripotent stem cells culture is achieved, the cells are passaged routinely every five to ten days using 1 mg/ml type IV collagenase (Gibco Invitrogen corporation products, San Diego, Calif., USA).

Culture Media:

Possibility one: Medium X, the medium consisting of 80% v/v DMEM\F12 or KO-DMEM, supplemented with 20% v/v defined fetal bovine serum (FBS) (HyClone, Utah, USA), 1 mM L-glutamine, 0.1 mM β-mercaptoethanol, 1% v/v non-essential amino acid stock (all from Gibco Invitrogen corporation products, San Diego, Calif., USA products).

Medium X can support the undifferentiated growth of bovine PSC that are cultured on feeder cells such as MEFs. However, if the bovine PSCs are cultured on MEFs with this medium and at high density (e.g., without passaging for at least 14 days), the bovine PSCs will undergo a spontaneous differentiation. In addition, if bovine PSCs are cultured with this medium on feeder-free culture systems the bovine PSCs will undergo a spontaneous differentiation.

Possibility 2: IL6RIL6 chimera medium, the cells were cultured using a medium consisting of 85% v/v DMEM\F12 (or KO-DMEM), supplemented with 15% v/v ko-serum replacement, 1 mM L-glutamine, 0.1 mM β-mercaptoethanol, 1% non-essential amino acid stock, 100 pg/ml TL6-TL6 receptor chimera (Chimera, biotest) and 50 ng/ml basic fibroblast growth factor (bFGF) (All products except for the IL6-IL6 receptor chimera are from Gibco Invitrogen corporation products, San Diego, Calif., USA). The cells were frozen in liquid nitrogen using a freezing solution consisting of 10% v/v DMSO (Sigma, St Louis, Mo., USA), 10% v/v FBS (Hyclone, Utah, USA) and 80% v/v DMEM\F12.

Possibility 3: Wnt3a medium, the cells were cultured using a medium consisting of 85% v/v DMEM\F12 (or KO-DMEM), supplemented with 15% v/v ko-serum replacement, 1 mM L-glutamine, 0.1 mM β-mercaptoethanol, 1% v/v non-essential amino acid stock, 10 ng\ml Wnt3a (Biotest), 100 ng/ml basic fibroblast growth factor (bFGF) and leukemia inhibitory factor (LIF) 3000 U\ml (All products, unless otherwise stated, are from Gibco Invitrogen corporation products, San Diego, Calif., USA). The cells were frozen in liquid nitrogen using a freezing solution consisting of 10% v/v DMSO (Sigma, St Louis, Mo., USA), 10% v/v FBS (Hyclone, Utah, USA) and 80% v/v DMEM/F12.

Blastocyst Cultivation from Horse:

Eight (8) days old embryo was obtained from Mare undergoing in uterus fertilization followed by uterus washing. Embryo was washed and maintained using Holding and transfer medium (BioLife, Item C15C, USA) till there were transferred to culture conditions (up to one hour).

Blastocysts can also be obtained from the following source:
Commercially available
In vitro fertilization (IVF), oocyte insemination in vitro
Nuclear Transfer (NT) of Horse cell.
Parthenogenesis

Derivation of Horse PSC Lines:

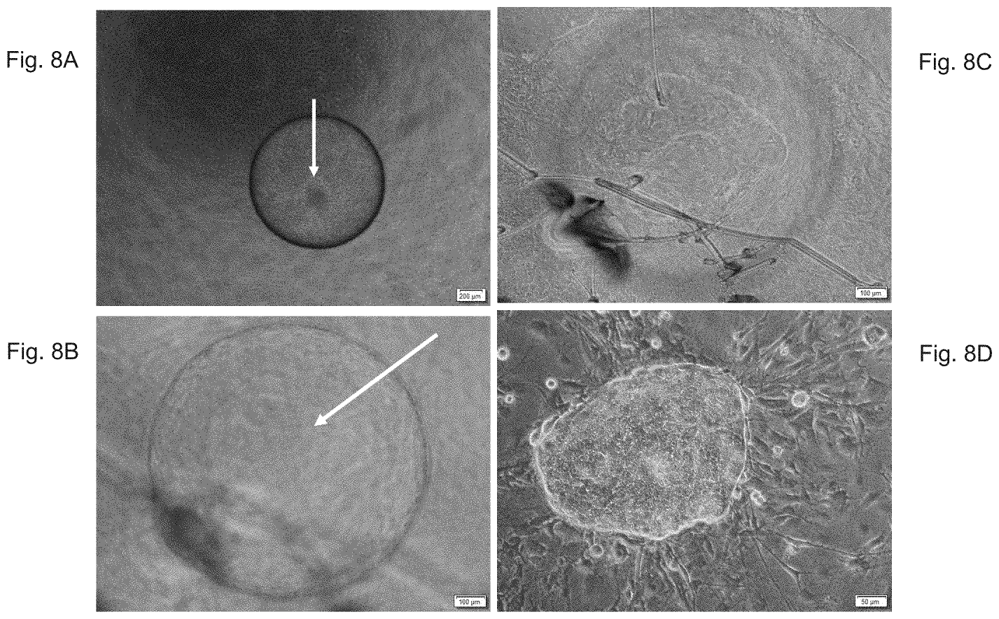

After zona pellucida digestion by Tyrode's acidic solution (Sigma Aldrich, St Louis, Mo., USA) the exposed blastocyst was plated. There are two plating possibilities: (i) on feeder layer, such as mitotically inactivated mouse embryonic fibroblasts (MEFs) or mitotically inactivated foreskin fibroblasts, (ii) on a suitable matrix (Matrigel™ matrix, Fibronectin, Laminin, Vitronectin, commercial cell matrices). The embryo was attached to the surface either by using 27 g needle, or by pulled Pasteur Pipette, or by covering the embryo by a drop of suitable matrix, or left overnight till the embryo spontaneously attached. Attached blastocysts are cultured on MEFs as whole embryos for 8-21 days post fertilization (e.g., at day 16 post insemination as shown in FIG. 8B for the specific embryo of HRS1) until a large cyst was developed. If needed due to the MEF or matrix quality, the embryos can be transferred in whole to new MEF-covered plates using 27 gouge syringe needles, leaving a few of the surrounding fibroblasts behind. After the embryo developed a cyst, a disc-like structure was isolated from it and plated separately on a fresh MEF or matrix-covered plate. Cells with stem cell morphology (small cells with large nucleus) were passaged mechanically. After a few passages (3-6 passages), when a homogenous culture was achieved, the cells were passaged routinely every five to ten days using 1 mg/ml type IV collagenase (Gibco Invitrogen corporation products, San Diego, Calif., USA).

Culture Media:

Medium X, as described hereinabove.

EB formation:

For the formation of Embryoid Bodies (EBs) two of four confluent wells in a four-well plate were used. The cells were left without splitting for 14 days to reach confluent culture, and EBs were spontaneously formed. Some remained attached to a culture surface and some were floating EBs (FIGS. 3A-B). EBs were grown using medium X.

Immunostaining:

Cells were fixed and exposed to the primary antibodies at room temperature. Then the cells were incubated with secondary antibodies. Table 1 below summarizes the reaction condition and antibodies.

TABLE 1
Immunostaining assays

| | | Indicating that the cells are |
|---|---|---|
| | | either pluripotent or |
| | Staining for the | differentiated cells of a specific |
| Immunostaining conditions | following antigens | embryonic germ layer |
| Fixation: 4% v/v PFA. | β III Tubulin | Differentiated ectoderm |
| Permeabilization buffer: 0.5% | (TUBB3) | |
| v/v Triton in PBS; | | |
| Blocking Buffer: 5% v/v Host | EOMES | Differentiated mesoderm |
| serum + 0.2% v/v Tween in | | |
| PBS; | | |
| Antibodies: | OCT4 | Undifferentiated pluripotent |
| β III Tubulin (TUBB3) (rabbit) | | stem cells |
| 1:100; | | |
| EOMES (mouse) 1:100; | | |
| OCT4 1:100; | | |
| Fixation: Methanol | Alpha 1 Fetoprotein | Differentiated endoderm |
| Permeabilization buffer: NA | | |
| Blocking Buffer: 5% v/v Host | | |
| serum + 0.2% v/v Tween in | α-Actinin | Differentiated mesoderm |
| PBS; | | |
| Antibodies: | | |
| Alpha 1 Fetoprotein (AFP) | | |
| (mouse) 1:200 | | |
| α-Actinin (ACTN1) (rabbit) | | |
| 1:100 | | |
| Fixation: Methanol | Nestin | Differentiated ectoderm |
| Permeabilization buffer: NA | | |
| Blocking Buffer: 5% v/v Host | | |
| serum + 0.2% v/v Tween in | α-Actinin | Differentiated mesoderm |
| PBS; | | |
| Antibodies: | | |
| Nestin (rabbit) 1:100 | | |
| α-Actinin (ACTN1) (mouse) | | |
| 1:100; | | |
| Fixation 100% cold Methanol, | TRA-1-60 | Undifferentiated pluripotent |
| incubation-5 min at room temp | Biolegend, Cat: | stem cells |
| | 330602; Dilution | |
| | 1:400 | |
| | Secondary Ab Goat | Undifferentiated pluripotent |
| | α-MOUSE IgM, | stem cells |
| | DyLight 550 | |
| | conjugated, dilution | |
| | 1:200 (Benthyl Lab) | |
| | TRA-1-81 | |
| | Biolegend, Cat: | |
| | 330702 | |
| | Dilution 1:100 | |
| | Secondary Ab Goat | |
| | α-MOUSE IgM, | |
| | DyLight 488 | |
| | conjugated, dilution | |
| | 1:100 (Benthyl Lab) | |

Table 1. Provided are the immunostaining conditions, antibodies used and the antigens which are identified by the antibodies. Also provided are the characteristics of the cells showing positive expression of the antigens. For example, OCT4 is a marker of undifferentiated pluripotent stem cells. “Host serum”—is a serum derived from the same species of host animal in which the antibody was produced. “NA”—not applicable.

Spontaneous differentiation into adipocytes—The bovine PSCs were cultured in “medium X” (which did not include dexamethasone) without cell passaging for 14-21 days, and were then fixed using paraformaldehyde for evaluation of lipid drops by Oil red staining.

Oil Red Staining:

Cells are fixed with paraformaldehyde (PFA) 4% v/v for 20 minutes (min) at room temperature (RT). After washing the PFA with phosphate buffered saline (PBS), the cells are incubated with Oil Red O solution (Sigma) for 10 minutes at RT. The culture is washed with water and visualized by phase contrast microscope.

### Example 1

### Derivation of Bovine Pluripotent Stem Cells from Extended Blastocysts

Experimental Results

While using the derivation method described in the “General Materials and Experimental Methods” section above, the present inventor demonstrate the derivation of 4 bovine cell lines (BVN1, BVN2, BVN5 and BVN6).

Derivation of Bovine Pluripotent Stem Cell Line BVN1:

The present inventor used two bovine embryos from day 7 post-fertilization, one of a defective pseudoblast, and one of a normal blastocyst. While the normal bovine embryo successfully grew in vitro on MEFs until day 13 post-fertilization, the defective embryo did not continue to develop in vitro. Accordingly the experiments continued with the normal blastocyst. Derivation of a bovine pluripotent stem cell line from an extended blastocyst—A bovine embryo from day 7 post-fertilization was cultured on MEFs in the presence of medium X as a whole embryo until day 13 post fertilization (i.e., 6 days in vitro on MEFs) until a large cyst was developed (FIGS. 1A-C).

After the embryo developed a cyst, a disc-like structure was isolated from the embryo and plated separately on a fresh MEF or on a matrix-coated plate (FIGS. 1A-C). At passage two colonies were transferred to the IL6RIL6 chimera medium and Matrigel™. Cells with stem cell morphology (small cells with large nucleus) were passaged mechanically. After a few passages (4-6 passages), when a population enriched with bovine pluripotent stem cells culture was achieved, the cells were passaged routinely every five to ten days using 1 mg/ml type IV collagenase (Gibco Invitrogen corporation products, San Diego, Calif., USA).

The resulting bovine pluripotent stem cell, termed “bPSC”, is the first bovine pluripotent stem cell which was obtained from an extended blastocyst culture technique and the first line was termed “BVN1”.

Bovine pluripotent stem cells exhibit a morphology similar to that of human ESC lines—As shown in FIGS. 1A-C, light microscopy revealed that the colonies formed by the bPSC presented similar morphology to that of hESC lines, e.g., a round colony with spaces between the cells and relatively large nucleus with distinct nucleoli.

Bovine pluripotent stem cells can be maintained on feeder cell layers with various culture media—As shown in FIGS. 2A-C, the bPSCs were cultured under different culture conditions and maintained the morphology of bPSC colonies. For example, bPSCs were cultured on feeder cells such as MEFs in the presence of a culture medium (“medium X”) which includes serum (FIG. 2A).

In addition, the bPSCs were also successfully cultured on MEFs in the presence of a serum-free culture medium such as the IL6RIL6 Chimera culture medium (FIG. 2C).

As shown in FIGS. 2A and 2C, bPSCs which were cultured on feeder cells exhibit typical spaces between the cells within the colony, and the cells exhibit a high nucleus to cytoplasm ratio, which is typical of pluripotent stem cells (PSC).

Bovine pluripotent stem cells can be maintained on feeder-free culture systems with a serum-free culture medium—The bPSCs were successfully cultured on feeder-free culture conditions, when cultured on a Matrigel™ matrix in the presence of a serum-free culture medium (the IL6RIL6 Chimera).

It is noted that bovine PSCs which were cultured with either a Wnt3a-containing medium or the IL6RIL6 Chimera medium while being passaged remained in the undifferentiated state (FIGS. 2A-C and data not shown).

Bovine pluripotent stem cells that were derived from an extended blastocyst exhibit a pluripotent cell phenotype—Immunostaining of bPSCs with the embryonic pluripotency marker Oct4 revealed positive staining (FIGS. 3A-B).

Bovine pluripotent stem cells that were derived from an extended blastocyst are capable of differentiation into embryoid bodies—The bPSCs were transferred to a four-well plate in the presence of medium X (consisting of 80% v/v DMEM\F12 or KO-DMEM, supplemented with 20% v/v defined fetal bovine serum). The cells were left without splitting for 14 days to reach confluent culture, and EBs were spontaneously formed. Some remained attached to a culture surface and some were floating EBs (FIGS. 4A-C).

The embryoid bodies that were generated from the bovine pluripotent stem cells include differentiated cells representative of all three embryonic germ layers—Immunostaining for differentiation markers demonstrated ability to differentiate to representative cells of the three embryonic germ layers (FIGS. 5A-D).

The bovine pluripotent stem cells are capable of spontaneous differentiation into adipocyte cells—The bovine PSCs were cultured on MEFs feeder layers in the presence of either medium X or the IL6RIL6 chimera medium, and spontaneously differentiated to adipocyte as background differentiation or when left without passaging for more than 14 days. The spontaneous differentiation was noted oil drops stained with Oil Red staining. It is noted that no forced induction to adipogenic lineage was performed. The medium used for the spontaneous differentiation did not include dexamethasone, which is a known inducer of stem cells towards the adipogenic lineage.

In sharp contrast to the bovine PSCs described herein the human delayed blastocysts cells which were described in WO2006/040763 never spontaneously differentiated into fat cells without induction via EBs formation or adipocytes differentiation medium and removal of MEFs feeder layer. As shown in FIGS. 6A-B spontaneous differentiation of bovine pluripotent cells into adipocytes (fat cells) revealed lipid droplets within the cells as stained with Oil Red staining. These results demonstrate the ability of the bPSCs to spontaneously differentiate into fat cells without the use of chemical or hormonal inductions.

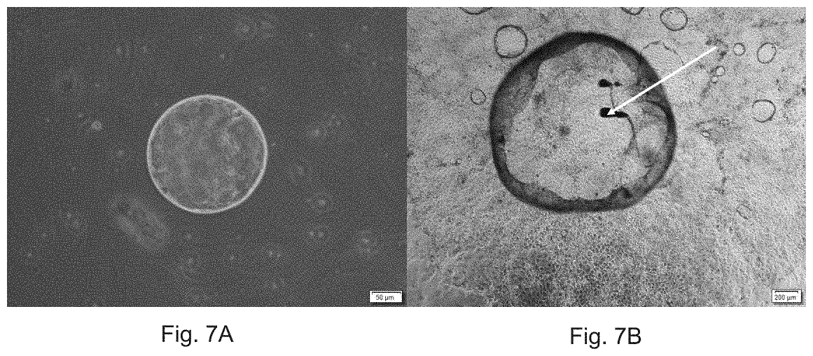

Derivation of bovine pluripotent stem cells from delayed bovine blastocyst line BVN6: Bovine blastocysts at 8 days post insemination were obtained and whole embryos were plated on feeder cells (mouse embryonic fibroblasts until day 16 post insemination (FIGS. 7A-B). As shown in FIG. 7B a notable cyst is developed on day 16 post insemination, and then a disc-like structure was isolated from the embryo and further plated separately on a fresh MEF. The cells were further cultured in a culture medium (medium X) for derivation of the bovine delayed blastocyst cell line.

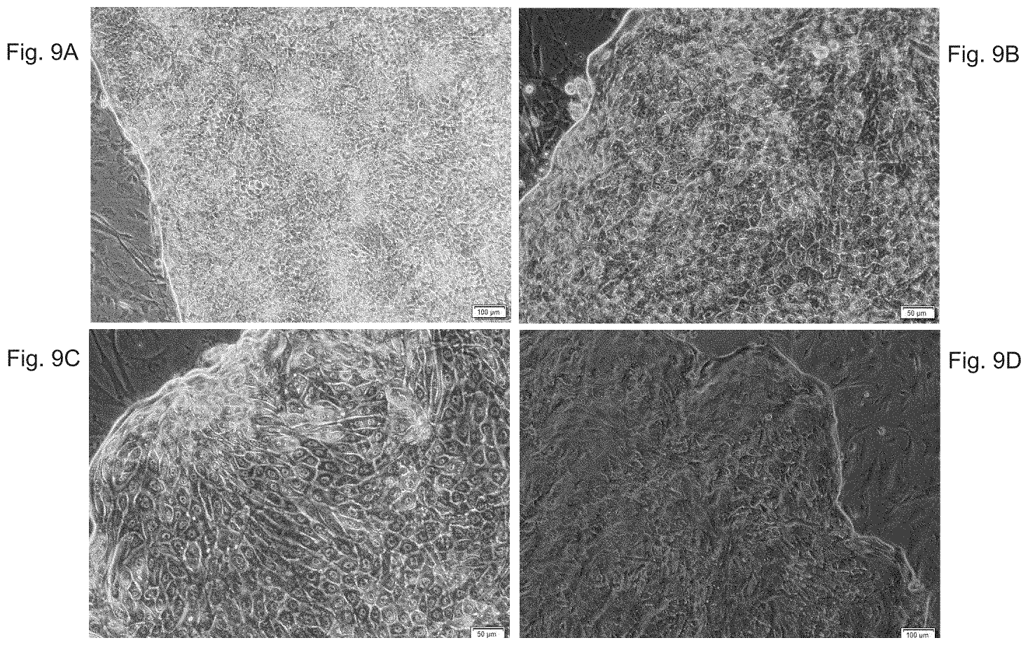

Morphological characterization of a bovine pluripotent stem cell (bPSCs) colony from lines BVN1, BVN2 and BVN5—As shown in FIGS. 9A-D the cells of the bPSC line BVN1, BVN2 and BVN5 maintain typical pluripotent stem cells morphology of small cells, each with a large nucleus at various passages (such as passages 8, 9 and 30), while being cultured in a culture medium such as medium X (e.g., up to 15 passages in medium X). For extended culturing and passages a culture medium comprising the IL6RIL6 chimera was used.

Expression of pluripotency markers TRA1-60 and TRA1-81 in bovine cell line BVN5 from delayed bovine blastocysts—FIGS. 10A-D show that the bPSC line BVN5 maintain the undifferentiated and pluripotent state when cultured in the presence of medium X for at least 8 passages, as is evidenced by positive staining of TRA1-60 (red) and TRA1-81 (green).

### Example 2

### Derivation of Horse Pluripotent Stem Cells from Extended Blastocysts

Experimental Results

While using the derivation method described in the “General Materials and Experimental Methods” section above, the present inventor demonstrate the derivation of 1 horse cell line (HRS1).

Derivation of a horse delayed blastocyst cell line—The present inventor used a horse embryos from day 8 post-insemination. As shown in FIG. 8A, a horse extended blastocysts with notable inner cell mass (ICM; white arrow) is observed at 8 days post insemination. The whole horse embryo was plated with mouse embryonic fibroblasts (MEFs) and was cultured in vitro in the presence of medium X until day 16 post insemination, when a notable cyst is developed (FIG. 8B). FIGS. 8B and 8C depict the same embryo at day 16 post insemination at different microscopic foci. At day 16 post insemination a disc-like structure was isolated from the embryo and further plated separately on a fresh MEF using medium X. As shown in FIG. 8D, the derived cells formed a colony of pluripotent stem cells characterized by small cells with large nucleus.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Additional References are Cited in Text

Bogliotti Y S, Wu J, Vilarino M, Okamur et al. Efficient derivation of stable primed pluripotent embryonic stem cells from bovine blastocysts. PNAS, 115 (9): 2090-2095, 2018.
Edwards R. G., Surani M. A. H. (1978). The primate blastocyst and its environment. Upsala Journal of Medical Sciences 22: 39-50.
Gardner R L. Investigation of cell lineage and differentiation in the extraembryonic endoderm of the mouse embryo. J. Embryological Experiment and Morphology 1982;
Mitalipova M, Beyham Z, and First N. Pluripotency of Bovine Embryonic Cell Line Derived from Precompacting Embryos Cloning, 3 (2):59-, 2001.
Reubinoff, B. E., Pera, M. F., Fong, C. Trounson, A., Bongso, A. (2000). Embryonic stem cell lines from human blastocysts: somatic differentiation in vitro. Nat. Biotechnol. 18, 399-404.
Thomson, J. A., Itskovitz-Eldor, J., Shapiro, S. S., Waknitz, M. A., Swiergiel, J. J., Marshall, V. S., Jones, J. M. (1998). Embryonic stem cell lines derived from human blastocysts. Science 282, 1145-1147 [erratum in Science (1998) 282, 1827].

What is claimed is:

1. A method of deriving a mammalian livestock pluripotent stem cells line, the method comprising:
(a) ex-vivo culturing a mammalian livestock embryo of at least 7 days post-fertilization for a culturing period of at least 4 days and no more than until 21 days post-fertilization so at to obtain an embryo comprising epiblast cell and/or late stage pluripotent stem cell;
(b) isolating from said embryo said epiblast cell and/or said late stage pluripotent stem cell, and
(c) culturing said epiblast cell and/or said late-stage pluripotent stem cell under conditions suitable for expansion of undifferentiated mammalian livestock pluripotent stem cells to thereby obtain a population of mammalian livestock pluripotent stem cells,
thereby deriving the mammalian livestock pluripotent stem cells line.

2. The method of claim 1, wherein said mammalian livestock pluripotent stem cells are capable of spontaneous differentiation into adipocytes in an absence of adipogenic differentiation agent(s).

3. The method of claim 2, wherein said mammalian livestock pluripotent stem cells are capable of spontaneous differentiation into adipocytes when cultured in a medium devoid of dexamethasone.

4. The method of claim 1, further comprising mechanically passaging said population of mammalian livestock pluripotent stem cells for at least 2 passages to thereby obtain a population enriched with said mammalian livestock pluripotent stem cells.

5. The method of claim 1, wherein said culturing said mammalian livestock embryo is performed on a two-dimensional culture system.

6. The method of claim 1, wherein said culturing said epiblast cell and/or said late-stage pluripotent stem cell is performed on a two-dimensional culture system, and optionally wherein said two-dimensional culture system comprises a feeder-free matrix.

7. The method of claim 1, wherein said culturing said mammalian livestock embryo is performed in a culture medium comprising a defined fetal mammalian livestock serum.

8. The method of claim 1, wherein said culturing said mammalian livestock embryo and wherein said culturing said epiblast cell and/or said late stage pluripotent stem cell is performed in a culture medium comprising the IL6RIL6 chimera.

9. The method of claim 8, wherein said culture medium further comprises basic fibroblast growth factor (bFGF).

10. The method of claim 1, wherein said culturing said mammalian livestock embryo and wherein said culturing said epiblast cell and/or said late stage pluripotent stem cell is performed in a culture medium comprising a Wnt3a polypeptide.

11. The method of claim 10, wherein said culture medium further comprising basic fibroblast growth factor (bFGF) and leukemia inhibitory factor (LIF).

12. The method of claim 1, wherein prior to said culturing said mammalian livestock embryo is covered with a drop of an extracellular matrix.

13. The method of claim 1, wherein cells of said population of mammalian livestock pluripotent stem cells spontaneously differentiate into adipogenic cell lineage when cultured without passaging for about 14-21 days in a culture medium.

14. The method of claim 13, wherein said culture medium comprises serum.

15. The method of claim 13, wherein said culture medium comprises the IL6RIL6 chimera.

16. An isolated mammalian livestock pluripotent stem cell generated by the method of claim 1, wherein said isolated mammalian livestock pluripotent stem cell is capable of differentiating into the ectoderm, mesoderm and ectoderm embryonic germ layers, and is capable of spontaneous differentiation into adipogenic cells when cultured in a medium devoid of dexamethasone.

17. A method of generating an adipocyte, comprising culturing the isolated mammalian livestock pluripotent stem cell of claim 16, in a culture medium devoid of chemical or hormonal induction towards adipogenic lineage for at least 10 days and no more than 60 days without passaging, thereby generating the adipocyte.

18. The method of claim 17, wherein said culture medium is devoid of dexamethasone.

19. A method of preparing food product, comprising incorporating the adipocyte generated by the method of claim 17 with a food product, thereby preparing the food product.

20. A food product comprising the adipocyte generated by the method of claim 17.
